(19) United States
(12) Patent Application Publication
(10) Pub. No.: US 2022/0079489 A1
(43) Pub. Date: Mar. 17, 2022

(54) METHOD FOR CONFIGURING PIN-OUT OF NONDIRECTIONAL USB TERMINAL ASSEMBLY FOR MEASURING ELECTROCARDIOGRAM AND ELECTROCARDIOGRAM MEASUREMENT DEVICE AND ELECTRODE DEVICE HAVING NONDIRECTIONAL USB TERMINAL WITH PIN-OUT CONFIGURED BY THE METHOD

(71) Applicant: Healthrian Co., Ltd., Daejeon (KR)

(72) Inventors: Tae-Hwan ROH, Daejeon (KR); Ki-Seok SONG, Daejeon (KR); Young-Ho CHOI, Daejeon (KR)

(21) Appl. No.: 17/122,288

(22) Filed: Dec. 15, 2020

(30) Foreign Application Priority Data
Sep. 15, 2020 (KR) 10-2020-0118161

(51) Int. Cl. A61B 5/273; A61B 5/28; A61B 5/339

(52) U.S. Cl. CPC A61B 5/273; A61B 5/339; A61B 5/28

(57) ABSTRACT

The present invention relates to a method for configuring pin-out of a nondirectional USB terminal assembly capable of measuring the electrocardiogram regardless of a connection direction between an electrocardiogram electrode device-side terminal and an electrocardiogram measurement device-side terminal and an electrocardiogram measurement device and an electrode device having a nondirectional USB terminal with pin-out configured by the method. The present invention relates to a result of research with the support of the Information Communication Planning and Evaluation Institute with the funding of the Korean government (Ministry of Science and Technology Information and Communication) in 2020 (Task number: 2020-0-00224 and task name: Development of Wireless Multi-channel ECG Device and Heart diagnosis Solution).

### CROSS-REFERENCE TO RELATED APPLICATIONS

This application claims the priority of Korean Patent Application No. 10-2020-0118161 filed on Sep. 15, 2020, in the Korean Intellectual Property Office, the disclosure of which is incorporated herein by reference.

### BACKGROUND OF THE INVENTION

### Field of the Invention

The present invention relates to a method for configuring pin-out of a nondirectional USB terminal assembly for measuring an electrocardiogram and an electrocardiogram measurement device and an electrode device having the nondirectional USB terminal with pin-out configured by the method, and more particularly, to a method for configuring pin-out of a nondirectional USB terminal assembly capable of measuring the electrocardiogram regardless of a connection direction between an electrocardiogram electrode device-side terminal and an electrocardiogram measurement device-side terminal and an electrocardiogram measurement device and an electrode device having a nondirectional USB terminal with pin-out configured by the method. The present invention relates to a result of research with the support of the Information Communication Planning and Evaluation Institute with the funding of the Korean government (Ministry of Science and Technology Information and Communication) in 2020 (Task number: 2020-0-00224 and task name: Development of Wireless Multi-channel ECG Device and Heart diagnosis Solution).

### Description of the Related Art

With the recent advancement of medical technology and an aging society, the expenditure of medical expenses is very high and to reduce the expenditure of such medical expenses, the existing treatment-oriented medical technology is gradually changing to diagnosis and prevention-oriented medical technology.

In particular, cardiovascular diseases are chronic diseases that are difficult to cure once the cardiovascular diseases occur, and it is very important to prevent the cardiovascular diseases by continuously measuring and managing cardiovascular conditions not only after the onset but before the onset.

Prior Art 1 (Korean Patent Registration No. 10-1002020) relates to a real-time ECG monitoring system and method, a patch-type ECG, and a communication device, and discloses a technology that can continuously measure, manage, and store 1-channel ECG for a long time using a patch-type ECG having three electrodes.

Prior Art 2 (Korean Patent Registration No. 10-1381136) is an extension of the number of ECG channels to 3 channels from the form of Prior Art 1 (Korean Patent Registration No. 10-1002020), and discloses a technology that can measure, manage, and store ECG of three channels more than Prior Art 1 (Korean Patent Registration No. 10-1002020) for more accurate cardiovascular diagnosis.

Prior Art 3 (Korean Patent Registration No. 10-1467351) relates to a 12-channel ECG electrode system, and discloses a wired electrode dividedly mounted on two sheets to measure a 12-channel ECG.

However, the related art (Prior Art 1 and Prior Art 2) provides a technology that can conveniently measure an electrocardiogram for a long time, but has a problem that the number of channels is limited to a maximum of 3 channels or less, so it is not possible to accurately diagnose cardiovascular conditions.

In addition, the related art (Prior Art 3) provides a technology that can measure the ECG up to 12 channels, but since the 12-channel electrode separated into two sheets is connected to an external ECG device with 10 lines, it is difficult to conveniently measure, store, and manage the 12-channel electrocardiogram for a long time, so that it is difficult to accurately diagnose the cardiovascular conditions.

For this reason, it is requested to develop a wearable ECG system capable of 1) long-term, continuous ECG measurement and 2) multi-channel (e.g., 12-channel) ECG measurement.

### SUMMARY OF THE INVENTION

The present invention has been made in an effort to provide a method for configuring pin-out of a nondirectional USB terminal assembly which can measure an electrocardiogram regardless of a connection direction between terminals of a wearable electrode device detecting an electrode signal of the electrocardiogram through a plurality of electrodes attachable to a body of a user and a measurement device that acquirers, displays, stores, and manages electrocardiogram information by receiving the electrode signal detected by the electrode device, respectively.

An exemplary embodiment of the present invention provides a method for configuring pin-out of a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, including: a data pin allocation step of allocating D+/D− for data communication with the external device to pins belonging to one pair among the pins of the electrocardiogram measurement device-side terminal; a power pin allocation step of allocating among pins of the measurement device-side terminal to which D+/D− is not allocated, the VBUS for power supply to the pins belonging to one pair; a pull-up pin allocation step of allocating among pins to which the D+/D− and the VBUS of the measurement device-side terminal are not allocated, a PULL-UP to any one pin; a ground pin allocation step of allocating GND to at least one of a pin located to face a pin to which the pull-up of the measurement device-side terminal is allocated and a pin located symmetrically to the pin to which the pull-up is allocated; a short pin allocation step of allocating, among the pins of the electrode device-side terminal, a pin corresponding to a pin to which the PULL-UP of the measurement device-side terminal is allocated and a pin corresponding to a pin to which the GND by a Short; a measurement device's electrode signal pin allocation step of allocating, among the pins of the measurement device-side terminal to which the D+/D−, VBUS, GND, and PULL-UP are not allocated, electrode signals for measuring the electrocardiogram; and an electrode device's electrode signal pin allocation step of allocating, among the pins of the electrode device-side terminal, the electrode signal to the pin corresponding to the pin to which the electrode signal of the measurement device-side terminal is allocated.

In this case, in the measurement device's electrode signal pin allocation step, among at least 6 pairs of pins that are not allocated any of the D+/D−, VBUS, GND, and determination signal (PULL-UP or PULL-DOWN) of the electrocardiogram measurement device-side terminal, electrode signals of V1 to V6 may be allocated to pins belonging to three pairs, any two electrode signals of RA, LA, and LL may be allocated to pins belonging to one pair, one remaining electrode signal which is not allocated among RA, LA, and LL may be allocated to the pins belonging to one pair, and the electrode signal of RL is allocated to the pins belonging to one pair, and in the electrode device's electrode signal pin allocation step, among the pins of the electrode device-side terminal, the electrode signals of V1 to V6, RA, RL, LA, and LL may be allocated to pins corresponding to the pins to which the electrodes signals of V1 to V6, RA, RL, LA, and LL of the measurement device-side terminal are allocated.

In this case, the measurement device-side terminal may be a USB Type-C female terminal (socket) in which pins A1 to A12 and pins B1 to B12 are arranged in two rows, the measurement device-side terminal may be a USB Type-C male terminal (plug) in which pins A1 to A12 and pins B1 to B12 are arranged in two rows, when the USB Type-C female terminal and the USB Type-C female terminal are connected in a forward direction, pins A1 to A12 and pins B1 to B12 of the USB Type-C female terminal and pins A1 to A12 and pins B1 to B12 of the USB Type-C female terminal may be shorted to correspond to each other in order, and when the USB Type-C female terminal and the USB Type-C female terminal are connected in a reverse direction, pins A1 to A12 and pins B1 to B12 of the USB Type-C female terminal and pins B1 to B12 and pins A1 to A12 of the USB Type-C female terminal may be shorted to correspond to each other in order.

In this case, in the data pin allocation step, D+ may be allocated to pins A6 and B6 diagonally located in the USB Type-C female terminal and D+ may be allocated to pins A7 and B7.

In this case, in the power pin allocation step, the VBUS may be allocated to pins A4, A9, B4, and B9 in the USB Type-C female terminal.

In this case, in the pull-up pin allocation step, the PULL-UP may be allocated to any one pin of pins A1, A12, B1, and B12 in the USB Type-C female terminal.

In this case, in the pull-up pin allocation step, the PULL-UP may be allocated to pin A1 in the USB Type-C female terminal, in the ground pin allocation step, the GND may be allocated to at least one of pin A12 or pin B12 in the USB Type-C female terminal, and in the short pin allocation step, pins A1 and A12 may be allocated by the short to each other or pins A1 and B12 may be allocated by the short to each other in the USB Type-C male terminal.

In this case, in the pull-up pin allocation step, the PULL-UP may be allocated to pin A12 in the USB Type-C female terminal, in the ground pin allocation step, the GND may be allocated to at least one of pin A1 or pin B1 in the USB Type-C female terminal, and in the short pin allocation step, pins A1 and A12 may be allocated by the short to each other or pins A12 and B1 may be allocated by the short to each other in the USB Type-C male terminal.

In this case, in the pull-up pin allocation step, the PULL-UP may be allocated to pin B1 in the USB Type-C female terminal, in the ground pin allocation step, the GND may be allocated to at least one of pin A12 or pin B12 in the USB Type-C female terminal, and in the short pin allocation step, pins A1 and B1 may be allocated by the short to each other or pins B1 and B12 may be allocated by the short to each other in the USB Type-C male terminal.

In this case, in the pull-up pin allocation step, the PULL-UP may be allocated to pin B12 in the USB Type-C female terminal, in the ground pin allocation step, the GND may be allocated to at least one of pin A1 or pin B1 in the USB Type-C female terminal, and in the short pin allocation step, pins A1 and B12 may be allocated by the short to each other or pins B1 and B12 may be allocated by the short to each other in the USB Type-C male terminal.

Further, another exemplary embodiment of the present invention provides an electrocardiogram measurement device having a terminal in which a pin is configured by a method for configuring pin-out of a nondirectional USB terminal assembly, including: an electrode signal input unit receiving electrode signal sensed by the electrocardiogram electrode device through a terminal on the side of the measurement device; a connection direction determination unit determining that a connection between the measurement device-side terminal and the electrode device-side terminal is in a forward direction when the PULL-UP is in a low state and determining that the connection between the measurement device-side terminal and the electrode device-side terminal is in a reverse direction when the PULL-UP is in a high state; an electrocardiogram information calculation unit acquiring electrocardiogram information by combining the electrode signals based on a determination result of the connection direction determination unit; and a display unit displaying the electrocardiogram information acquired by the electrocardiogram information calculation unit.

Further, yet another exemplary embodiment of the present invention provides a method for configuring pin-out of a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, including: a data pin allocation step of allocating D+/D− for data communication with the external device to pins belonging to one pair among the pins of the electrocardiogram measurement device-side terminal; a ground pin allocation step of allocating, among pins of the measurement device-side terminal to which D+/D− is not allocated, the GND to the pins belonging to one pair; a pull-up pin allocation step of allocating, among pins of the measurement device-side terminal to which the D+/D− and the GND are not allocated, a PULL-DOWN to any one pin; a power pin allocation step of allocating VBUS for power supply to at least one of a pin located to face a pin to which the pull-down of the measurement device-side terminal is allocated and a pin located symmetrically to the pin to which the pull-down is allocated; a short pin allocation step of among the pins of the electrode device-side terminal, a pin corresponding to a pin to which the PULL-DOWN of the measurement device-side terminal is allocated and a pin corresponding to a pin to which the VBUS is allocated by a Short; a measurement device's electrode signal pin allocation step of allocating, among the pins of the measurement device-side terminal to which the D+/D−, VBUS, GND, and PULL-DOWN are not allocated, electrode signals for measuring the electrocardiogram; and an electrode device's electrode signal pin allocation step of allocating, among the pins of the electrode device-side terminal, the electrode signal to the pin corresponding to the pin to which the electrode signal of the measurement device-side terminal is allocated.

In this case, in the measurement device's electrode signal pin allocation step, among at least 6 pairs of pins that are not allocated any of the D+/D−, VBUS, GND, and PULL-DOWN of the electrocardiogram measurement device-side terminal, electrode signals of V1 to V6 may be allocated to pins belonging to three pairs, any two electrode signals of RA, LA, and LL may be allocated to pins belonging to one pair, one remaining electrode signal which is not allocated among RA, LA, and LL may be allocated to the pins belonging to one pair, and the electrode signal of RL may be allocated to the pins belonging to one pair, and in the electrode device's electrode signal pin allocation step, among the pins of the electrode device-side terminal, the electrode signals of V1 to V6, RA, RL, LA, and LL may be allocated to pins corresponding to the pins to which the electrodes signals of V1 to V6, RA, RL, LA, and LL of the measurement device-side terminal are allocated.

In this case, the measurement device-side terminal may be a USB Type-C female terminal (socket) in which pins A1 to A12 and pins B1 to B12 are arranged in two rows, the measurement device-side terminal may be a USB Type-C male terminal (plug) in which pins A1 to A12 and pins B1 to B12 are arranged in two rows, when the USB Type-C female terminal and the USB Type-C female terminal are connected in a forward direction, pins A1 to A12 and pins B1 to B12 of the USB Type-C female terminal and pins A1 to A12 and pins B1 to B12 of the USB Type-C female terminal may be shorted to correspond to each other in order, and when the USB Type-C female terminal and the USB Type-C female terminal are connected in a reverse direction, pins A1 to A12 and pins B1 to B12 of the USB Type-C female terminal and pins B1 to B12 and pins A1 to A12 of the USB Type-C female terminal may be shorted to correspond to each other in order.

In this case, in the data pin allocation step, D+ may be allocated to pins A6 and B6 diagonally located in the USB Type-C female terminal and D+ may be allocated to pins A7 and B7.

In this case, in the ground power pin allocation step, the GND may be allocated to pins A1, A12, B1, and B12 in the USB Type-C female terminal.

In this case, in the pull-down pin allocation step, the PULL-DOWN may be allocated to any one pin of pins A4, A9, B4, and B9 in the USB Type-C female terminal.

In this case, in the pull-down pin allocation step, the PULL-DOWN may be allocated to pin B12 in the USB Type-C female terminal, in the power pin allocation step, the VBUS may be allocated to at least one of pin A9 or pin B9 in the USB Type-C female terminal, and in the short pin allocation step, pins A4 and A9 may be allocated by the short to each other or pins A4 and B9 may be allocated by the short to each other in the USB Type-C male terminal. In this case, in the pull-down pin allocation step, the PULL-DOWN may be allocated to pin A9 in the USB Type-C female terminal, in the power pin allocation step, the VBUS may be allocated to at least one of pin A4 or pin B4 in the USB Type-C female terminal, and in the short pin allocation step, pins A4 and A9 may be allocated by the short to each other or pins A9 and B4 may be allocated by the short to each other in the USB Type-C male terminal.

In this case, in the pull-down pin allocation step, the PULL-DOWN may be allocated to pin B4 in the USB Type-C female terminal, in the power pin allocation step, the VBUS may be allocated to at least one of pin A9 or pin B9 in the USB Type-C female terminal, and in the short pin allocation step, pins A9 and B4 may be allocated by the short to each other or pins B4 and B9 may be allocated by the short to each other in the USB Type-C male terminal.

In this case, in the pull-down pin allocation step, the PULL-DOWN may be allocated to pin B9 in the USB Type-C female terminal, in the power pin allocation step, the VBUS may be allocated to at least one of pin A4 or pin B4 in the USB Type-C female terminal, and in the short pin allocation step, pins A4 and B9 may be allocated by the short to each other or pins B4 and B9 may be allocated by the short to each other in the USB Type-C male terminal.

Further, still yet another exemplary embodiment of the present invention provides an electrocardiogram measurement device having a terminal in which a pin is configured by a method for configuring pin-out of a nondirectional USB terminal assembly, including: an electrode signal input unit receiving electrode signal sensed by the electrocardiogram electrode device through a terminal on the side of the measurement device; a connection direction determination unit determining that a connection between the measurement device-side terminal and the electrode device-side terminal is in a forward direction when the PULL-DOWN is in a high state and determining that the connection between the measurement device-side terminal and the electrode device-side terminal is in a reverse direction when the PULL-DOWN is in a low state; an electrocardiogram information calculation unit acquiring electrocardiogram information by combining the electrode signals based on a determination result of the connection direction determination unit; and a display unit displaying the electrocardiogram information acquired by the electrocardiogram information calculation unit.

Further, still yet another exemplary embodiment of the present invention provides a method for configuring pin-out of a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, including: a data pin allocation step of allocating D+/D− for data communication with the external device to pins belonging to one pair among the pins of the electrocardiogram measurement device-side terminal; a first power/ground pin allocation step of allocating, among pins to which D+/D− of the measurement device-side terminal is not allocated, any one of VBUS for power supply and GND to the pins belonging to one pair; a determination signal pin allocation step of allocating a determination signal for determining the connection direction between the measurement device-side terminal and the electrode device-side terminal to at least one pin among pins to which the D+/D− and any one of the VBUS and the GND of the measurement device-side terminal are not allocated; a second power/ground pin allocation step of allocating the remaining one which is not allocated of the VBUS and the GND to at least one of a pin located to face the pin to which the determination signal of the measurement device-side terminal is allocated and a pin located symmetrically to the pin to which the determination signal is allocated; a short pin allocation step of allocating, among the pins of the electrode device-side terminal, a pin corresponding to the pin to which the determination signal of the measurement device-side terminal is allocated and a pin corresponding to the pin allocated in the second power/ground pin allocation step by a Short; a measurement device's electrode signal pin allocation step of allocating, among the pins of the measurement device-side terminal to which the D+/D−, VBUS, GND, and PULL-UP are not allocated, electrode signals for measuring the electrocardiogram; and an electrode device's electrode signal pin allocation step of allocating, among the pins of the electrode device-side terminal, the electrode signal to the pin corresponding to the pin to which the electrode signal of the measurement device-side terminal is allocated.

Further, still yet another exemplary embodiment of the present invention provides an electrocardiogram measurement device having a terminal in which a pin is configured by a method for configuring pin-out of a nondirectional USB terminal assembly, including: an electrode signal input unit receiving electrode signal sensed by the electrocardiogram electrode device through a terminal on the side of the measurement device; a connection direction determination unit determining whether a connection between the measurement device-side terminal and the electrode device-side terminal is in a forward direction or a reverse direction based on the determination signal; an electrocardiogram information calculation unit acquiring electrocardiogram information by combining the electrode signals based on a determination result of the connection direction determination unit; and a display unit displaying the electrocardiogram information acquired by the electrocardiogram information calculation unit.

Further, still yet another exemplary embodiment of the present invention provides an electrocardiogram electrode device having a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, including: an electrode module constituted by a plurality of electrodes attachable to a body of a user; and a nondirectional USB terminal electrically connected to an electrocardiogram measurement device for transmitting electrode signal sensed through each of the plurality of electrodes, in which in the electrode device-side terminal, pins corresponding to pins of the measurement device-side terminal, respectively are arranged in two rows, at least two pins among the pins of the electrode device-side terminal are shorted to each other, and the electrode signals are allocated to pins which are not shorted to each other among the pins of the electrode device-side terminal.

According to an exemplary embodiment of the present invention, there is a technical effect of accurately, conveniently, and continuously measuring, displaying, storing, and managing an electrocardiogram for a long time.

Further, according to an exemplary embodiment of the present invention, a technical effect of measuring an electrocardiogram regardless of a connection direction between terminals of a wearable electrode device detecting an electrode signal of the electrocardiogram through a plurality of electrodes attachable to a body of a user and a measurement device that acquirers, displays, stores, and manages electrocardiogram information by receiving the electrode signal detected by the electrode device, respectively.

Further, according to an exemplary embodiment of the present invention, there is a technical effect that a measurement device that acquires, displays, stores, and manages the electrocardiogram information can receive, analyze, and process the electrode signal detected by the wearable electrode device through a nondirectional USB (USB C type) terminal which follows a pin-out configuration of a standard, and transmit measurement data to an external device (desktop, notebook, tablet, etc.) that can support the nondirectional USB (USB C type) terminal or can be charged by a power adapter.

### BRIEF DESCRIPTION OF THE DRAWINGS

FIG. 1 is a diagram illustrating an overall configuration of a wearable electrocardiogram system with a nondirectional USB terminal according to the present invention.

FIGS. 2A and 2B are diagrams for describing a structure of a terminal provided in an electrocardiogram measuring device according to the present invention.

FIGS. 3A and 3B are diagrams for describing a structure of a terminal provided in an electrocardiogram electrode device according to the present invention.

FIGS. 4A and 4B are diagrams illustrating a standard pin-out configuration of a USB C type terminal.

FIG. 5, FIG. 6A, FIG. 6B, FIG. 7, FIG. 8A, FIG. 8B, FIG. 9, FIG. 10A, FIG. 10B, FIG. 11, FIG. 12A and FIG. 12B are diagrams for describing setting of a pin-out function of a nondirectional USB terminal assembly according to the present invention using pull-up.

FIG. 13, FIG. 14A, FIG. 14B, FIG. 15, FIG. 16A, FIG. 16B, FIG. 17, FIG. 18A, FIG. 18B. FIG. 19, FIG. 20A and FIG. 20B are diagrams for describing setting of a pin-out function of a nondirectional USB terminal assembly according to the present invention using pull-down.

FIGS. 21A and 21B are diagrams for describing assignment of electrode signals to pins of a terminal in the present invention.

FIG. 22, FIG. 23A and FIG. 23B are block diagrams for describing a configuration and an operation of a wearable electrocardiogram measurement device with a nondirectional USB terminal according to the present invention.

FIG. 24, FIG. 25A and FIG. 25B are flowcharts for describing a method for configuring pin-out of a nondirectional USB terminal assembly for measuring an electrocardiogram according to the present invention.

### DETAILED DESCRIPTION OF THE PREFERRED EMBODIMENT

The present invention will be described below in detail with reference to the accompanying drawings. Herein, the repeated description and the detailed description of publicly-known function and configuration that may make the gist of the present invention unnecessarily ambiguous will be omitted. Embodiments of the present invention are provided for more completely describing the present invention to those skilled in the art. Accordingly, shapes, sizes, and the like of elements in the drawings may be exaggerated for clearer explanation.

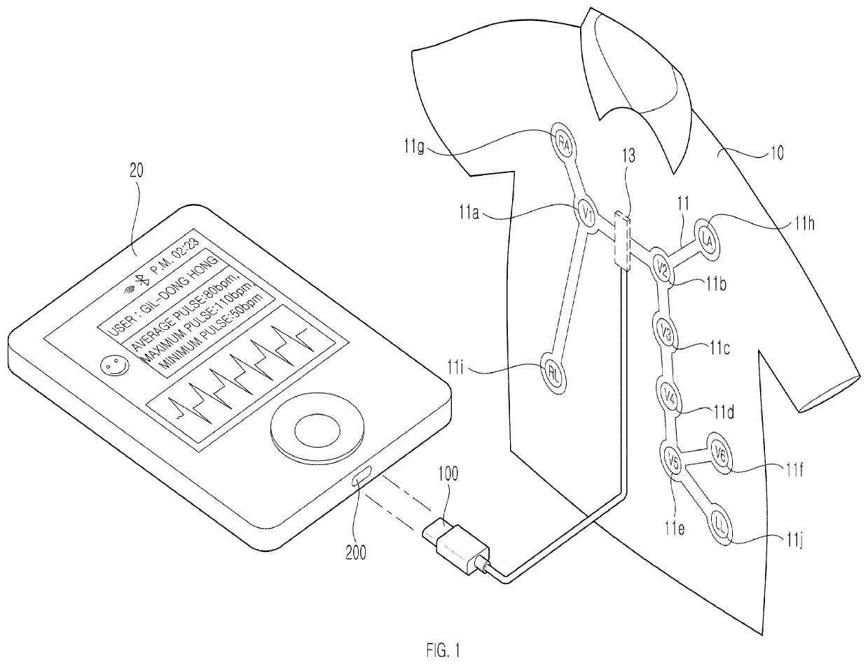

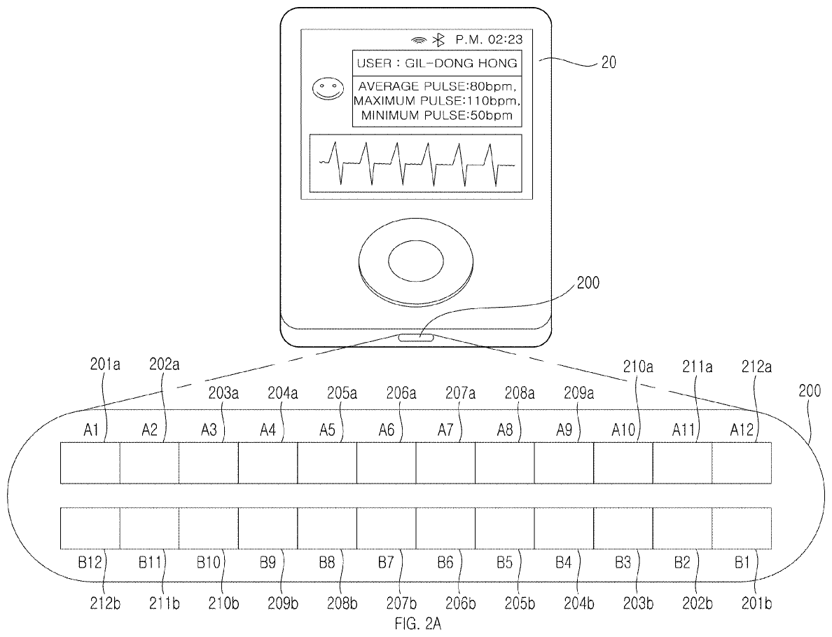

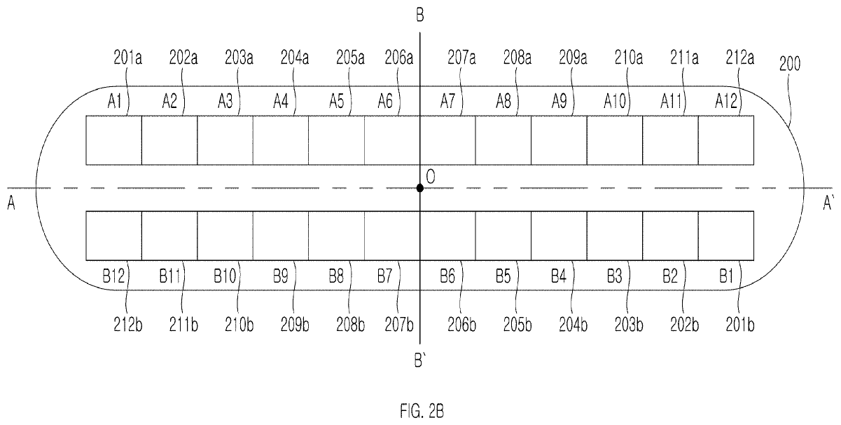

FIG. 1 is a diagram illustrating an overall configuration of a wearable electrocardiogram system with a nondirectional USB terminal according to the present invention.

Referring to FIG. 1, an electrocardiogram measuring system having a nondirectional USB terminal according to the present invention includes an electrocardiogram electrode device 10 and an electrocardiogram measurement device 20.

Here, the electrocardiogram electrode device 10 may be provided with an electrode module constituted by a plurality of electrodes 11a to 11j in which a plurality of electrodes are attached to each part of the body of a user (e.g., a patient), and may be implemented in the form of wearable clothing which the user may wear. The electrode module constituted by the plurality of electrodes 11a to 11j provided in the electrocardiogram electrode device 10 may be implemented as one electrode sheet 11 which is a patch form that is collectively formed on one sheet so as to be collectively attached to the body of the user. The electrocardiogram electrode device 10 collects electrode signals related to the electrocardiogram of the user detected through the plurality of electrodes 11a to 11j and transfers the electrode signals to the electrocardiogram measurement device 20 through electrical connection between the terminals 100 and 200. On the other hand, it is preferable that the plurality of electrodes 11a to 11j are connected with a plurality of pins of the terminal 100 by separate cables, respectively, and in some cases, the plurality of electrodes 11a to 11j may Be configured to be separately connected to the plurality of pins of the terminal 100 through a cable passing through a hub 13.

Hereinafter, in the present invention, an electrocardiogram measurement system will be described in a case where the number of electrocardiogram channels to be measured is 12. However, the present invention is not particularly limited to the case of measuring an electrocardiogram of 12 channels, but may also be applied to a case of measuring an electrocardiogram of n channels having two or more channels.

In order to measure the 12-channel ECG, 10 electrodes 11a to 11j are required, but positions when the 10 electrodes 11a to 11j are arranged are not absolutely fixed, and the positions may be determined body-structurally. Table 1 below shows respective bodies of the body of the patient to which 10 electrodes constituting an integrated 12-channel ECG dedicated electrode sheet 10 are attached as an example.

TABLE 1

| Electrode | Electrode position |
|---|---|
| V1 | just next to the right sternum in 4th intercostal space |
| | (between ribs 4 and 5) |
| V2 | just next to the left sternum in 4th intercostal space |
| | (between ribs 4 and 5) |
| V3 | between V2 and V4 |
| V4 | median clavicle line in the fifth intercostal space |
| | (between ribs 5 and 6) |
| V5 | Horizontally parallel to V4, left side of the front armpit |
| V6 | Horizontally parallel to V4 and V5, middle armpit line |
| RA | Avoid the thick muscles above the right arm. |
| LA | Placed on the left arm similarly as being placed on the right |
| | arm. |
| RL | next to the calf muscle in the right leg |
| LL | Placed on the left leg similarly as being placed on the right |
| | leg. |

There is no standard proposal for an absolute position for the arrangement of 12-channel electrocardiogram electrodes commonly used by all subjects, and 10 electrodes may be disposed at slightly different positions depending on a body shape of a person to be measured. However, in order to minimize the size of the electrode for the convenience of the person to be measured, for example, based on a square formed by the first to fourth electrodes (V1 to V4; 11a to 11d) after consulting a circulatory internal medicine, limb electrodes (RA, LA, RL, LL; 11g, 11h, 11i, 11j) are preferably located in the upper right (RA), upper left (LA), lower right (RL), and lower left (LL), respectively. That is, since the limb electrodes (RA, LA, RL, LL; 11g, 11h, 11i, 11j) are generally located closer to the first to sixth electrodes (V1 to V6; 11a to 11f) than the electrode arrangement of the 12-channel electrocardiogram measured in a hospital, it is necessary to locate the limb electrodes on the right arm, left arm, right leg, and left leg, not the chest, as shown in Table 1 for more accurate 12-channel ECG measurement.

On the other hand, the electrocardiogram measurement device 20 serves to receive the electrode signals (e.g., V1 to V6, RA, LA, RL, and LL) collected through the electrocardiogram electrode device 10 between the terminals 100 and 200, and extract the user's cardiovascular state information (for example, current heart rate, average heart rate, maximum heart rate, minimum heart rate, instantaneous heart rate, etc.) by analyzing the 12-channel electrocardiogram.

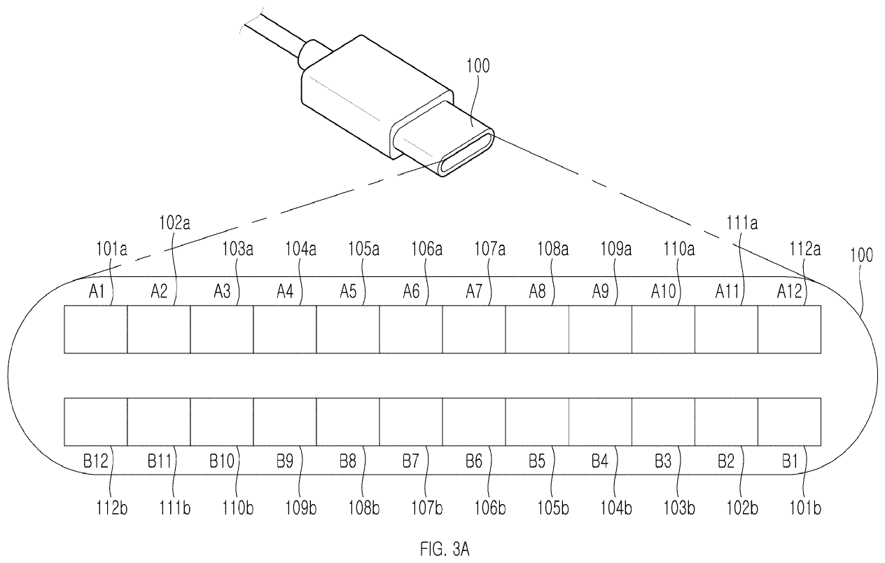

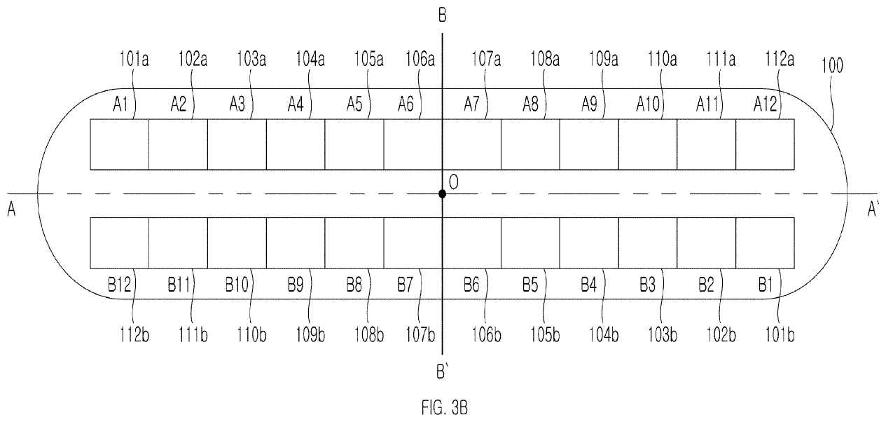

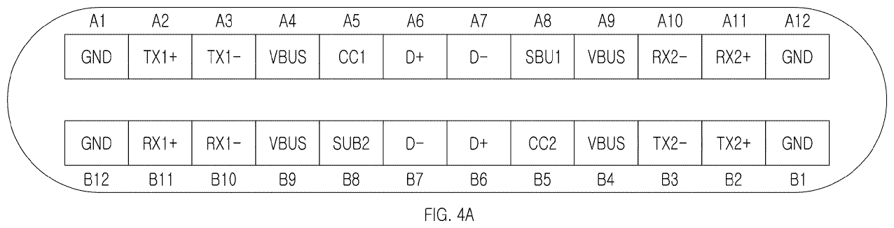

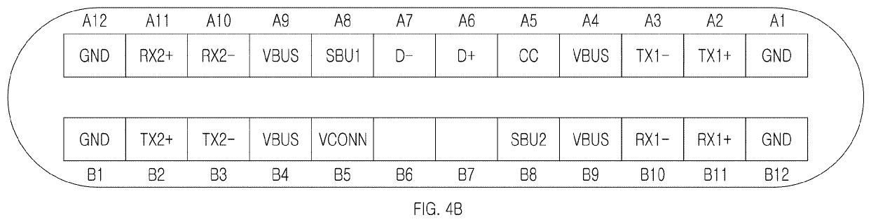

FIGS. 2 and 3 are diagrams for describing structures of terminals 100 and 200 provided in an electrocardiogram electrode device 10 and an electrocardiogram measurement device 20, respectively according to the present invention.

First, referring to FIGS. 2A and 2B, the ECG measurement device 20 includes a terminal 200 electrically connected to the terminal 100 on the side of the ECG electrode device 10. More specifically, the terminal 200 on the side of the electrocardiogram measurement device 20 as a nondirectional USB terminal may be implemented as a female terminal (socket) as shown in FIG. 2A. However, in some cases, the terminal 200 on the side of the electrocardiogram measurement device 20 may be implemented as a male terminal (plug). Here, the nondirectional USB terminal means a USB terminal in which short pins 201a to 212a; 201b to 212b are arranged in two rows and the female terminal and the male terminal may be connected to each other regardless of up/down directions (forward/reverse direction) and the type of USB terminal will be referred to as the nondirectional USB terminal. As the nondirectional USB terminal means a USB terminal in which the short pins 201a to 212a; 201b to 212b are arranged in two rows and the female terminal and the male terminal may be connected to each other regardless of the up/down directions as illustrated in FIG. 2A, a widely known USB C type (Type-C) terminal is representatively used. The terminal 200 on the side of the electrocardiogram measurement device 20 according to the present invention as the USB Type-C terminal is preferably is a USB Type-C female terminal (socket) in which pins A1 to A12 201a to 212a and pins B1 to B12 201b to 212b are arranged in two rows as illustrated in FIG. 2A. Referring to FIG. 2B, in the terminal 200 on the side of the electrocardiogram measurement device 20, with two pins (e.g., pin A1 and pin B1) located diagonally to each other with respect to a terminal center O as a pair, pins A1 to A12 201a to 212a and pins B1 to B12 201b to 212b are arranged in two rows. At this time, pins A1 to A12 201a to 212a and pins B1 to B12 2 01b to 212b are located to face each other based on a horizontal line (A-A′) passing through the terminal center O. In addition, pins A1 to A6 201a to 206a and pins A7 to A12 207a to 212a are located symmetrically with respect to a vertical line (B-B′) passing through the terminal center O, while pins B1 to B6 201b to 206b and pins B7 to B12 207b to 212b are located symmetrically to each other with respect to the vertical line (B-B′). For example, pin A1 201a is located to face pin B12 212b, and is located symmetrically to pin B1 201b.

Referring to FIGS. 3A and 3B, the electrocardiogram electrode device 10 includes a terminal 100 electrically connected to the terminal 200 on the side of the electrocardiogram measurement device 20. The terminal 100 on the side of the electrocardiogram electrode device 10 is implemented as a nondirectional USB terminal that may be connected to the electrocardiogram measurement device 20 side terminal 200 described above. More specifically, the electrocardiogram measurement device 10 side terminal 100 may be implemented as the nondirectional USB terminal may be implemented as the male terminal (plug) as illustrated in FIG. 3A. However, in some cases, the electrocardiogram electrode device 10 side terminal 100 may be implemented as the female terminal (socket). That is, when the electrocardiogram measurement device 20 side terminal 200 is implemented as the female terminal (socket), the electrocardiogram electrode device 10 side terminal 100 is implemented as the male terminal (plug) and when the electrocardiogram measurement device 20 side terminal 200 is implemented as the male terminal (plug), the electrocardiogram electrode device 10 side terminal 100 is implemented as the female terminal (socket). The electrocardiogram electrode device 10 side terminal 100 as the USB Type-C terminal is preferably a USB Type-C male terminal (plug) in which pins A1 to A12 101a to 112a and pins B1 to B12 101b to 112b are arranged in two rows as illustrated in FIGS. 3A and 3B. In this case, when the electrocardiogram electrode device 10 side USB Type-C male terminal 100 is connected to the electrocardiogram measurement device 20 side USB Type-C female terminal 200 in a forward direction, pins A1 to A12 101a to 112a and pins B1 to B12 101b to 112b of the terminal 100 are correspondingly connected to pins A1 to A12 201a to 212a and pins B1 to B12 201b to 212b of the terminal 200, respectively in order. On the other hand, when the electrocardiogram electrode device 10 side USB Type-C male terminal 100 is connected to the electrocardiogram measurement device 20 side USB Type-C female terminal 200 in a reverse direction, pins B1 to B12 101b to 112b and pins A1 to A12 101a to 112a of the terminal 100 are correspondingly connected to pins A1 to A12 201a to 212a and pins B1 to B12 201b to 212b of the terminal 200, respectively in order.

Hereinafter, it will be described that a nondirectional USB terminal assembly (male terminal/female terminal) constituted by the electrocardiogram electrode device 10 side terminal 100 and the electrocardiogram measurement device 20 side terminal 200 corresponds to a widely known USB Type-C terminal assembly (male terminal/female terminal). However, the present invention is not particularly limited to the USB Type-C terminal assembly and if two pins which are located diagonally to each other are arranged in two rows with one pair, and as a result, the female terminal and the male terminal may be connected to each other regardless an up/down direction, the nondirectional USB terminal assembly may be implemented as any nondirectional USB terminal assembly other than the USB Type-C terminal assembly.

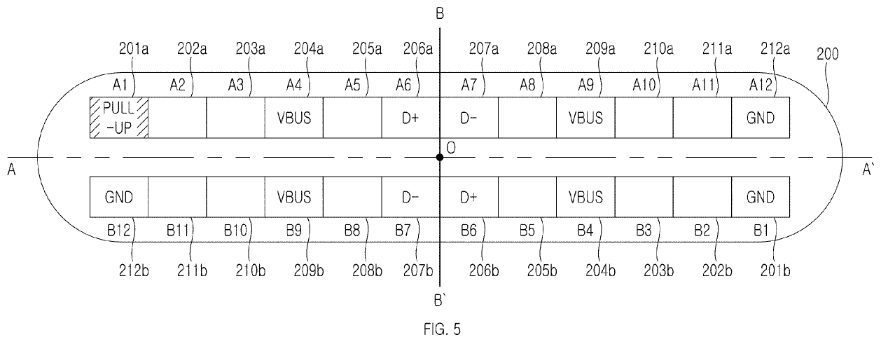

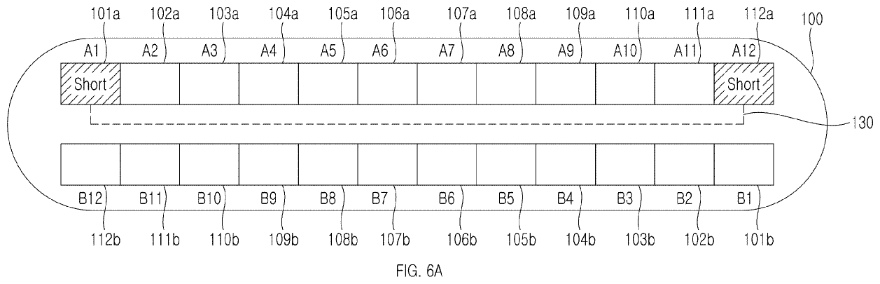

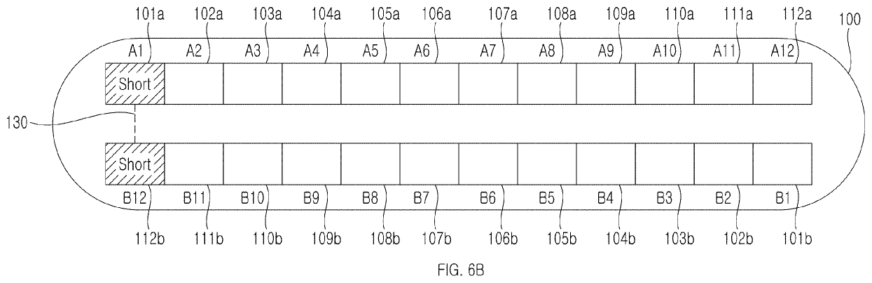

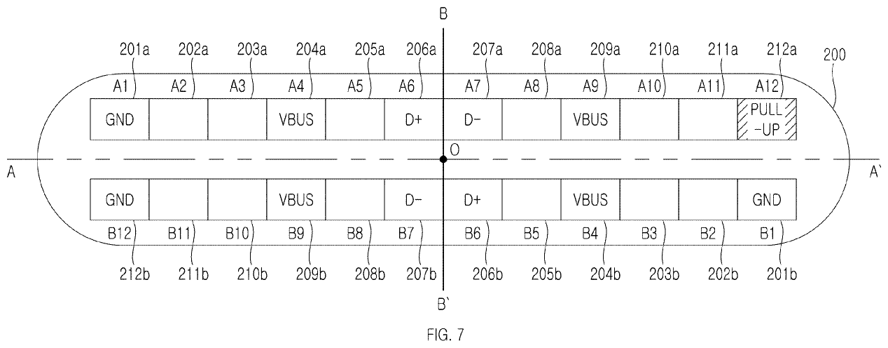

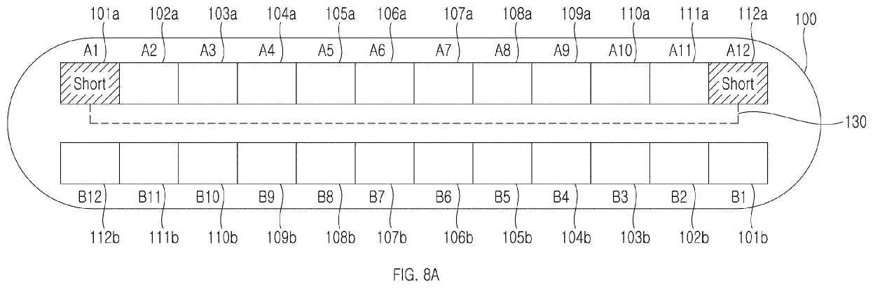

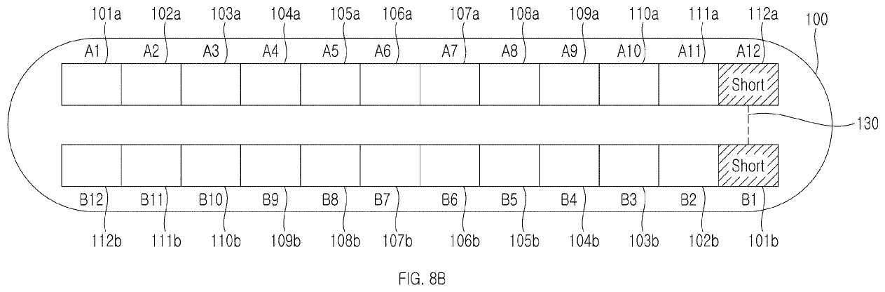

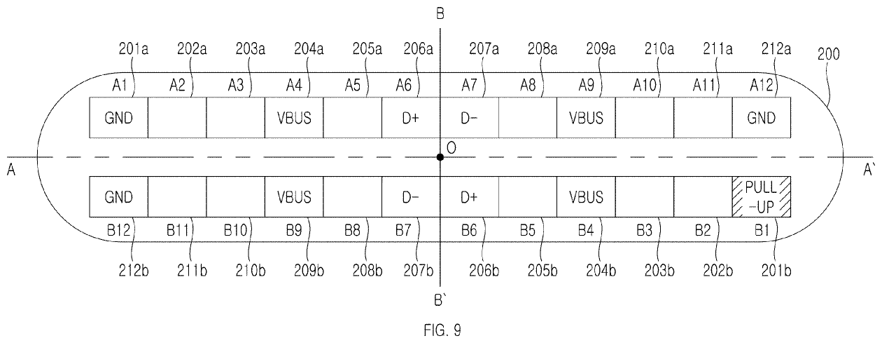

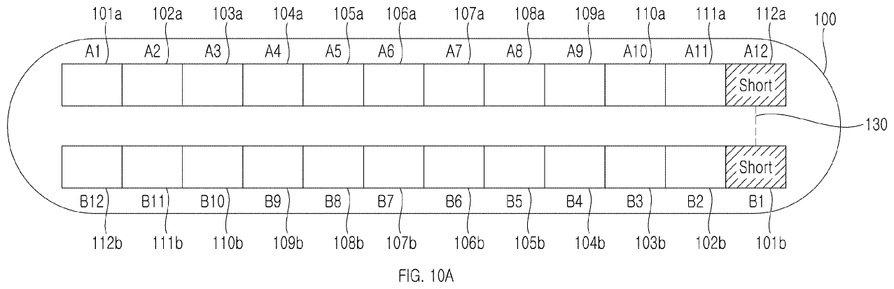

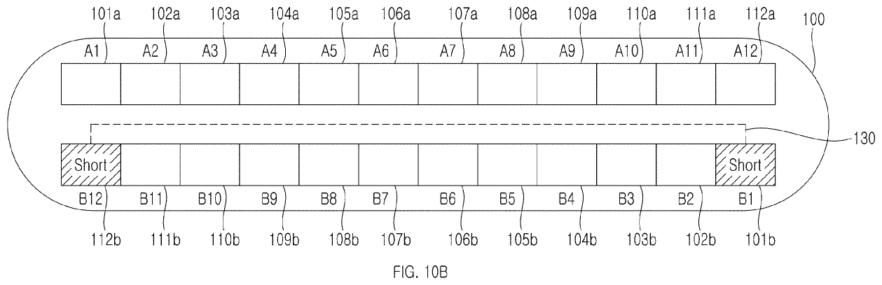

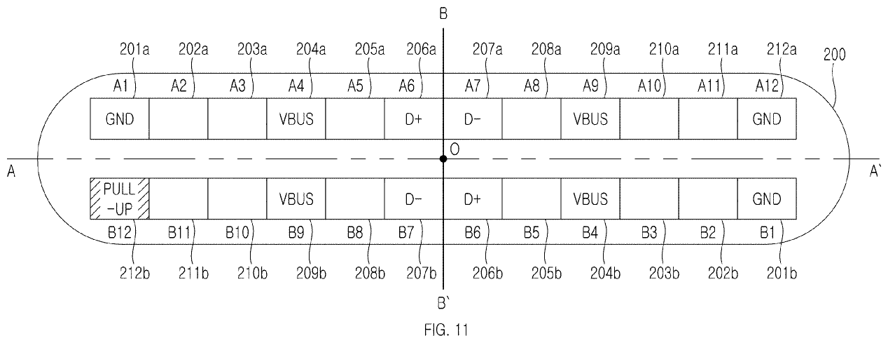

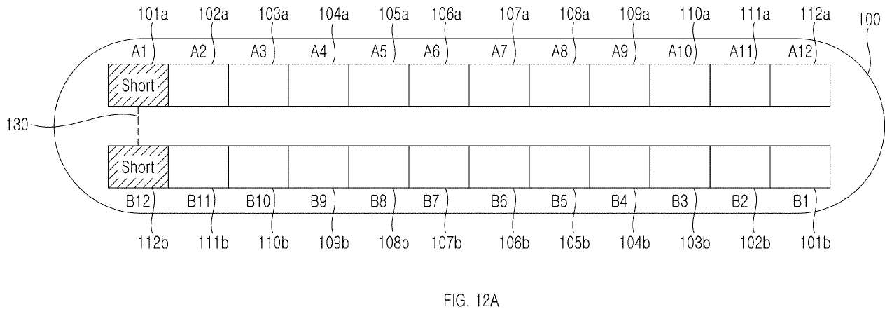

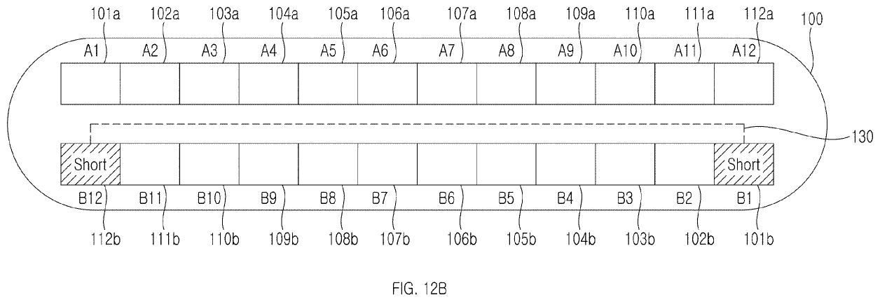

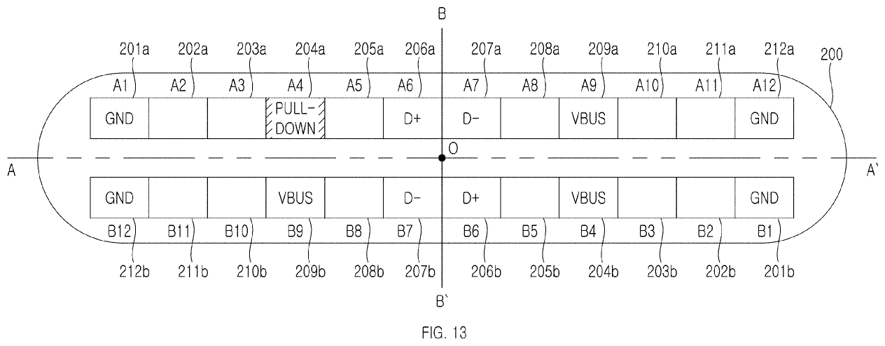

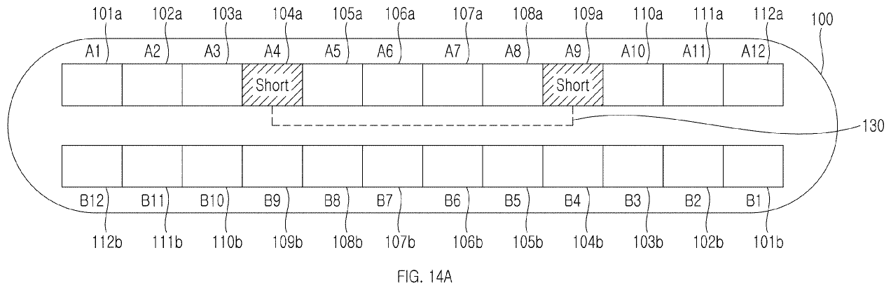

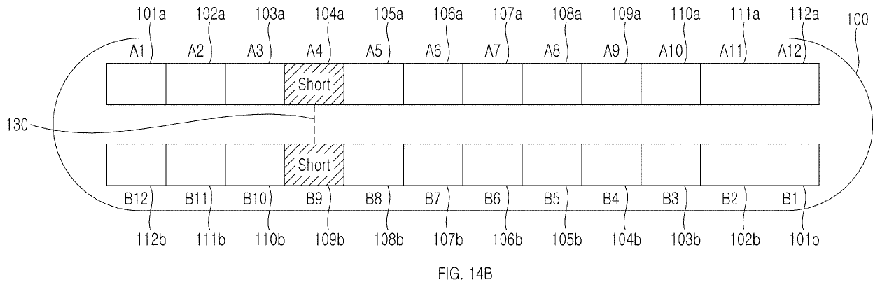

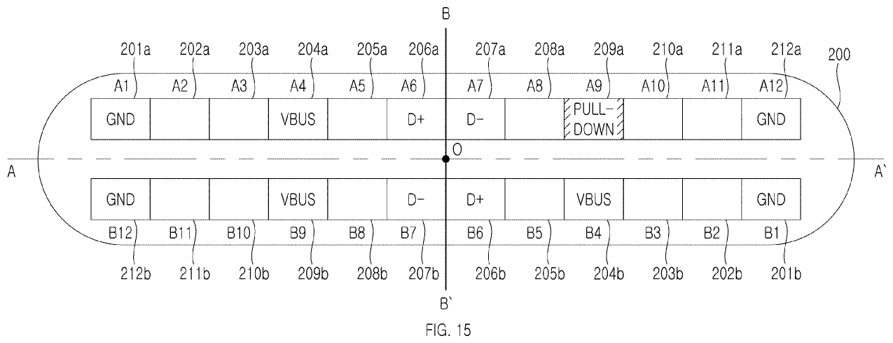

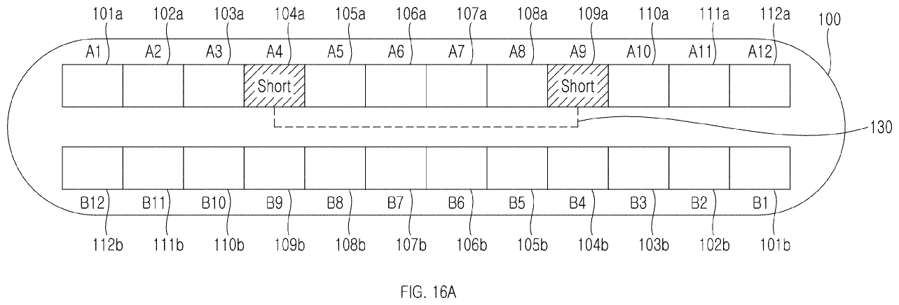

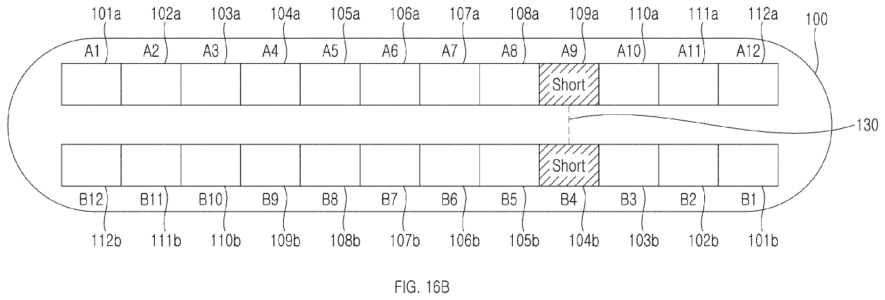

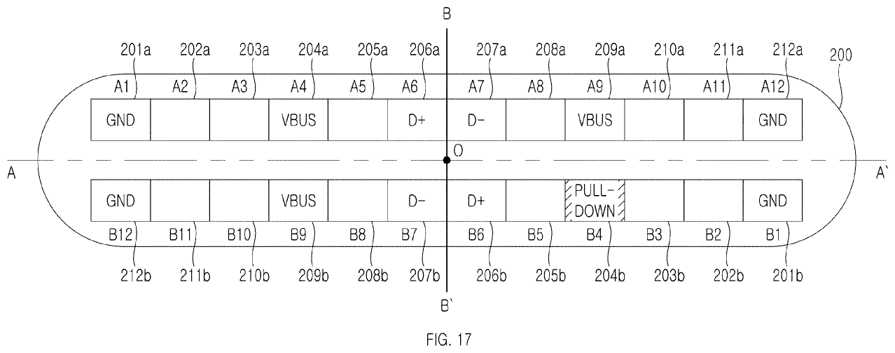

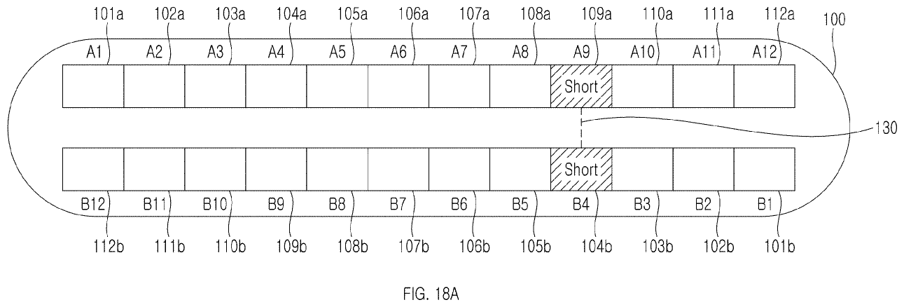

FIGS. 4 to 21 are diagrams for describing setting of a pin-out function of a nondirectional USB terminal assembly according to the present invention. In particular, setting the pin-out function of the nondirectional USB terminal assembly according to the present invention may be divided into setting the pin-out function using pull-up to be described later with reference to FIGS. 5 to 12 and setting the pin-out function using pull-down to be described later with reference to FIGS. 13 to 20. Here, the pull-up or the pull-down is an input signal value which becomes a basis for determining whether the connection between the terminal 100 and the terminal 200 in the electrocardiogram measuring device 20 is forward or reverse as described later and since the pull-up or the pull-down corresponds to a signal for determining directionality of the connection between the terminals in the present invention, the pull-up or the pull-down will be referred to as a determination signal.

The electrocardiogram measurement device 20 side terminal 200 according to the present invention preferably follows a standard pin-out configuration of the USB Type-C female terminal (socket) illustrated in FIG. 4A in order to transmit measurement data to an external device (desktop, notebook, tablet, etc.) supporting the nondirectional USB type, in particular, USB Type-C terminal and have an advantage of versatility which is chargeable through a power adapter supporting the USB Type-C terminal. Further, the electrocardiogram electrode device 10 side terminal 100 according to the present invention preferably follows the standard pin-out configuration of the USB Type-C male terminal (plug) illustrated in FIG. 4B so as to be connected to the electrocardiogram measurement device 20 side terminal 200 which follows the standard pin-out configuration of the USB Type-C female terminal (socket) as described above.

Hereinafter, a pin-out configuration will be described, which is used for measuring the electrocardiogram regardless of the connection direction (forward direction/reverse direction) between the electrocardiogram electrode device 10 side terminal 100 and the electrocardiogram measurement device 20 side terminal 200 by using the pull-up with reference to FIGS. 5 to 12.

First, D+/D− for data communication with the external device is allocated to pins belonging to one pair among the pins of the electrocardiogram measurement device 20 side terminal 200. When the terminal 200 is the USB Type-C female terminal (socket), D+(Dp1), D—(Dn1), D+(Dp2), and D—(Dn2) are allocated to pin A6 pin 206a, pin A7 207a, pin B6 206b, and pin B7 207b, respectively as illustrated in FIG. 5 so as to follow the standard pin-out configuration of the USB Type-C female terminal (socket). In addition, among pins to which D+ and D− of the measurement device 20 side terminal 200 are not allocated, VBUS for power supply is allocated to the pins belonging to one pair. When the terminal 200 is the USB Type-C female terminal (socket), the VBUS is allocated to pin A4 204a, pin A9 209a, pin B4 204b, and pin B9 209b as illustrated in FIG. 5 so as to follow the standard pin-out configuration of the USB Type-C female terminal (socket).

Among pins to which D+ and D− of the measurement device 20 side terminal 200 and the VBUS are not allocated, the pull-up is allocated to any one pin. When the terminal 200 is the USB Type-C female terminal (socket), the pull-up is allocated to pin A1 201a (Exemplary embodiment 1), allocated to pin A12 212a (Exemplary embodiment 2), allocated to pin B1 201b (Exemplary embodiment 3), and allocated to pin B12 212b (Exemplary embodiment 4) in the present invention. Here, the pull-up as a configuration for inputting a stable high (1) value as a signal value by solving an unstable floating state in which an input signal value is neither high (1) nor low (0) and since the function of the pull-up is widely known, a detailed description thereof will be omitted.

In addition, GND is allocated to at least one of a pin located to face a pin to which the pull-up of the measurement device 20 side terminal 200 is allocated and a pin located symmetrically to the pin to which the pull-up of the measurement device 20 side terminal 200 is allocated.

In the case of Exemplary embodiment in which the pull-up is allocated to pin A1 201a of the terminal 200 as illustrated in FIG. 5 according to the standard pin configuration of the USB Type-C female terminal (socket), GND is allocated to at least one of pin A12 212a and pin B12 212b. More specifically, in the case of Exemplary embodiment 1, the GND may be allocated only to one of pins A12 212a and B12 212b, the GND may be allocated to two pins (pins A12 and B1; pins A12 and B12) including pin A12 212a, or the GND may be allocated to two pins (pins A12 and B12; pins B1 and B12) including pin B12 (212b), and as illustrated in FIG. 5, the GND may be allocated to all of pins A12 212a, B1 201b, and B12 212b. In this case, among the pins of the electrode device 10 side terminal 100, the GND is allocated to Short for electrically connecting a pin corresponding to a pin to which the pull-up of the measurement device 20 side terminal 200 is allocated and a pin corresponding to a pin to which the GND is allocated to each other. Here, the corresponding pin means a connected pin when being connected between the terminals in the forward direction. More specifically, when the GND is allocated to pin A12 212a of the terminal 200, pins A1 101a and A12 112a of the terminal 100 are connected to each other by a short 130 as illustrated in FIG. 6A and when the GND is allocated to pin B12 212b of the terminal 200, pins A1 101a and B12 112b of the terminal 100 are connected to each other by the short 130 as illustrated in FIG. 6B. When the GND is allocated to pin B1 201b of the terminal 200 in Exemplary embodiment 1, pins A1 101a and B1 101b of the terminal 100 should not be connected to each other by the short. In this case, when pins A1 101a and B1 101b of the terminal 100 are connected to each other by the short, if the terminal 100 and the terminal 200 are connected in the forward direction or connected in the reverse direction, the signal value of the PULL-UP is not distinguished and the PULL-UP similarly has a low (0) signal value.

In the case of Exemplary embodiment 2 in which the PULL-UP is allocated to pin A12 212a of the terminal 200 as illustrated in FIG. 7 according to the standard pin configuration of the USB Type-C female terminal (socket), the GND is allocated to at least one of pin A1 201a and pin B1 201b. More specifically, in the case of Exemplary embodiment 2, the GND may be allocated only to any one of pin A1 201a and B1 201b, the GND may be allocated to two pins (pins A1 and B1; pins A1 and B12) including pin A1 201a or the GND may be allocated to two pins (pins A1 and B1; pins B1 and B12) including pin B1 (201b), and as illustrated in FIG. 7, the GND may be allocated to all of pins A1 201a, B1 201b, and B12 212b. In this case, among the pins of the electrode device 10 side terminal 100, a pin corresponding to a pin to which the PULL-UP of the measurement device 20 side terminal 200 is allocated and a pin corresponding to a pin to which the GND is allocated are allocated by the Short. More specifically, when the GND is allocated to pin A1 201a of the terminal 200, pins A1 101a and A12 112a of the terminal 100 are connected to each other by the short 130 as illustrated in FIG. 8A and when the GND is allocated to pin B1 201b of the terminal 200, pins A12 112a and B1 101b of the terminal 100 are connected to each other by the short 130 as illustrated in FIG. 8B. When the GND is allocated to pin B12 212b of the terminal 200 in Exemplary embodiment 2, pins A12 112a and B12 112b of the terminal 112 should not be connected to each other by the short. In this case, when pins A12 112a and B12 112b of the terminal 100 are connected to each other by the short, if the terminal 112 and the terminal 200 are connected in the forward direction or connected in the reverse direction, the signal value of the PULL-UP is not distinguished and the PULL-UP similarly has the low (0) signal value.

In the case of Exemplary embodiment 3 in which the PULL-UP is allocated to pin B1 201b of the terminal 200 as illustrated in FIG. 9 according to the standard pin configuration of the USB Type-C female terminal (socket), the GND is allocated to at least one of pin A12 212a and pin B12 212b. More specifically, in the case of Exemplary embodiment 3, the GND may be allocated only to one of pins A12 212a and B12 212b, the GND may be allocated to two pins (pins A1 and A12; pins A12 and B12) including pin A12 212a, or the GND may be allocated to two pins (pins A1 and B12; pins A12 and B12) including pin B12 (212b), and as illustrated in FIG. 9, the GND may be allocated to all of pins A1 201a, A12 212a, and B12 212b. In this case, among the pins of the electrode device 10 side terminal 100, a pin corresponding to a pin to which the PULL-UP of the measurement device 20 side terminal 200 is allocated and a pin corresponding to a pin to which the GND is allocated are allocated by the Short. More specifically, when the GND is allocated to pin A12 212a of the terminal 200, pins A12 112a and B1 101b of the terminal 100 are connected to each other by the short 130 as illustrated in FIG. 10A and when the GND is allocated to pin B12 212b of the terminal 200, pins B1 101b and B12 112b of the terminal 100 are connected to each other by the short 130 as illustrated in FIG. 10B. When the GND is allocated to pin A1 201a of the terminal 200 in Exemplary embodiment 3, pins A1 101a and B1 101b of the terminal 100 should not be connected to each other by the short. In this case, when pins A1 101a and B1 101b of the terminal 100 are connected to each other by the short, if the terminal 100 and the terminal 200 are connected in the forward direction or connected in the reverse direction, the signal value of the PULL-UP is not distinguished and the PULL-UP similarly has the low (0) signal value.

In the case of Exemplary embodiment 3 in which the PULL-UP is allocated to pin B12 212b of the terminal 200 as illustrated in FIG. 11 according to the standard pin configuration of the USB Type-C female terminal (socket), the GND is allocated to at least one of pin A1 201a and pin B1 201b. More specifically, in the case of Exemplary embodiment 4, the GND may be allocated only to one of pins A1 201a and B1 201b, the GND may be allocated to two pins (pins A1 and A12; pins A1 and B1) including pin A1 201a, or the GND may be allocated to two pins (pins A1 and B1; pins A12 and B1) including pin B1 (201b), and as illustrated in FIG. 11, the GND may be allocated to all of pins A1 201a, A12 212a, and B1 201b. In this case, among the pins of the electrode device 10 side terminal 100, a pin corresponding to a pin to which the PULL-UP of the measurement device 20 side terminal 200 is allocated and a pin corresponding to a pin to which the GND is allocated are allocated by the Short. More specifically, when the GND is allocated to pin A1 201a of the terminal 200, pins A1 101a and B12 112b of the terminal 100 are connected to each other by the short 130 as illustrated in FIG. 12A and when the GND is allocated to pin B1 201b of the terminal 200, pins B1 101b and B12 112b of the terminal 100 are connected to each other by the short 130 as illustrated in FIG. 12B. When the GND is allocated to pin A12 212a of the terminal 200 in Exemplary embodiment 4, pins A12 112a and B12 112b of the terminal 100 should not be connected to each other by the short. In this case, when pins A12 112a and B12 112b of the terminal 100 are connected to each other by the short, if the terminal 112 and the terminal 200 are connected in the forward direction or connected in the reverse direction, the signal value of the PULL-UP is not distinguished and the PULL-UP similarly has the low (0) signal value.

Hereinafter, a pin-out configuration will be described, which is used for measuring the electrocardiogram regardless of the connection direction (forward direction/reverse direction) between the electrocardiogram electrode device 10 side terminal 100 and the electrocardiogram measurement device 20 side terminal 200 by using the pull-down with reference to FIGS. 13 to 20.

First, D+/D− for data communication with the external device is allocated to pins belonging to one pair among the pins of the electrocardiogram measurement device 20 side terminal 200. When the terminal 200 is the USB Type-C female terminal (socket), D+(Dp1), D—(Dn1), D+(Dp2), and D—(Dn2) are allocated to pin A6 pin 206a, pin A7 207a, pin B6 206b, and pin B7 207b, respectively as illustrated in FIG. 13 so as to follow the standard pin-out configuration of the USB Type-C female terminal (socket). In addition, among pins to which D+ and D− of the measurement device 20 side terminal 200 are not allocated, the GND is allocated to the pins belonging to one pair. When the terminal 200 is the USB Type-C female terminal (socket), the GND is allocated to pin A1 201a, pin A12 212a, pin B1 201b, and pin B12 212b as illustrated in FIG. 13 so as to follow the standard pin-out configuration of the USB Type-C female terminal (socket).

Among pins to which D+ and D− of the measurement device 20 side terminal 200 and the GND are not allocated, the pull-down is allocated to any one pin. When the terminal 200 is the USB Type-C female terminal (socket), the pull-down is allocated to pin A4 204a (Exemplary embodiment 5), allocated to pin A9 209a (Exemplary embodiment 6), allocated to pin B4 204b (Exemplary embodiment 7), and allocated to pin B9 209b (Exemplary embodiment 8) in the present invention. Here, the pull-down as a configuration for inputting a stable low (0) value as a signal value by solving an unstable floating state in which an input signal value is neither high (1) nor low (0) and since the function of the pull-down is widely known, a detailed description thereof will be omitted.

In addition, the VBUS for power supply is allocated to at least one of a pin located to face a pin to which the pull-down of the measurement device 20 side terminal 200 is allocated and a pin located symmetrically to the pin to which the pull-down is allocated.

In the case of Exemplary embodiment 5 in which the PULL-DOWN is allocated to pin A4 204a of the terminal 200 as illustrated in FIG. 13 according to the standard pin configuration of the USB Type-C female terminal (socket), the VBUS is allocated to at least one of pin A9 209a and pin B9 209b. More specifically, in the case of Exemplary embodiment 5, the VBUS may be allocated only to one of pins A9 209a and B9 209b, the VBUS may be allocated to two pins (pins A9 and B4; pins A9 and B9) including pin A9 209a, or the VBUS may be allocated to two pins (pins A9 and B9; pins B4 and B9) including pin B9 (209b), and as illustrated in FIG. 5, the VBUS may be allocated to all of pins A9 209a, B4 204b, and B9 209b. In this case, among the pins of the electrode device 10 side terminal 100, a pin corresponding to a pin to which the PULL-DOWN of the measurement device 20 side terminal 200 is allocated and a pin corresponding to a pin to which the VBUS is allocated are allocated by the Short. Here, the corresponding pin means a connected pin when being connected between the terminals in the forward direction. More specifically, when the VBUS is allocated to pin A9 209a of the terminal 200, pins A4 104a and A9 109a of the terminal 100 are connected to each other by the short 130 as illustrated in FIG. 14A and when the VBUS is allocated to pin B9 209b of the terminal 200, pins A4 104a and B9 109b of the terminal 100 are connected to each other by the short 130 as illustrated in FIG. 14B. When the VBUS is allocated to pin B4 204b of the terminal 200 in Exemplary embodiment 5, pins A4 104a and B4 104b of the terminal 104 should not be connected to each other by the short. In this case, when pins A4 104a and B4 104b of the terminal 100 are connected to each other by the short, if the terminal 100 and the terminal 200 are connected in the forward direction or connected in the reverse direction, the signal value of the PULL-DOWN is not distinguished and the PULL-down similarly has the high (1) signal value.

In the case of Exemplary embodiment 6 in which the PULL-DOWN is allocated to pin A9 209a of the terminal 200 as illustrated in FIG. 15 according to the standard pin configuration of the USB Type-C female terminal (socket), the VBUS is allocated to at least one of pin A4 204a and pin B4 204b. More specifically, in the case of Exemplary embodiment 6, the VBUS may be allocated only to one of pins A4 204a and B4 204b, the VBUS may be allocated to two pins (pins A4 and B4; pins A4 and B9) including pin A4 204a, or the VBUS may be allocated to two pins (pins A4 and B4; pins B4 and B9) including pin B4 (204b), and as illustrated in FIG. 15, the VBUS may be allocated to all of pins A4 204a, B4 204b, and B9 209b. In this case, among the pins of the electrode device 10 side terminal 100, a pin corresponding to a pin to which the PULL-DOWN of the measurement device 20 side terminal 200 is allocated and a pin corresponding to a pin to which the VBUS is allocated are allocated by the Short. More specifically, when the VBUS is allocated to pin A4 204a of the terminal 200, pins A4 104a and A9 109a of the terminal 100 are connected to each other by the short 130 as illustrated in FIG. 16A and when the VBUS is allocated to pin B4 204b of the terminal 200, pins A9 109a and B4 104b of the terminal 100 are connected to each other by the short 130 as illustrated in FIG. 16B. When the VBUS is allocated to pin B9 209b of the terminal 200 in Exemplary embodiment 6, pins A9 109a and B9 109b of the terminal 109 should not be connected to each other by the short. In this case, when pins A9 109a and B9 109b of the terminal 100 are connected to each other by the short, if the terminal 100 and the terminal 200 are connected in the forward direction or connected in the reverse direction, the signal value of the PULL-DOWN is not distinguished and the PULL-DOWN similarly has the high (1) signal value.

In the case of Exemplary embodiment 7 in which the PULL-DOWN is allocated to pin B4 204b of the terminal 200 as illustrated in FIG. 17 according to the standard pin configuration of the USB Type-C female terminal (socket), the VBUS is allocated to at least one of pin A9 209a and pin B9 209b. More specifically, in the case of Exemplary embodiment 7, the VBUS may be allocated only to any one of pins A9 209a and B9 209b, the VBUS may be allocated to two pins (pins A4 and A9; pins A9 and B9) including pin A9 209a, or the VBUS may be allocated to two pins (pins A4 and B9; pins A9 and B9) including pin B9 (209b), and as illustrated in FIG. 17, the VBUS may be allocated to all of pins A4 204a, A9 209a, and B9 209b. In this case, among the pins of the electrode device 10 side terminal 100, a pin corresponding to a pin to which the PULL-DOWN of the measurement device 20 side terminal 200 is allocated and a pin corresponding to a pin to which the VBUS is allocated are allocated by the Short. More specifically, when the VBUS is allocated to pin A9 209a of the terminal 200, pins A9 109a and B4 104b of the terminal 100 are connected to each other by the short 130 as illustrated in FIG. 18A and when the VBUS is allocated to pin B9 209b of the terminal 200, pins B4 104b and B9 109b of the terminal 100 are connected to each other by the short 130 as illustrated in FIG. 18B. When the VBUS is allocated to pin A4 204a of the terminal 200 in Exemplary embodiment 7, pins A4 104a and B4 104b of the terminal 100 should not be connected to each other by the short. In this case, when pins A4 104a and B4 104b of the terminal 100 are connected to each other by the short, if the terminal 100 and the terminal 200 are connected in the forward direction or connected in the reverse direction, the signal value of the PULL-DOWN is not distinguished and the PULL-DOWN similarly has the high (1) signal value.

In the case of Exemplary embodiment 8 in which the PULL-DOWN is allocated to pin B9 209b of the terminal 200 as illustrated in FIG. 19 according to the standard pin configuration of the USB Type-C female terminal (socket), the VBUS is allocated to at least one of pin A4 204a and pin B4 204b. More specifically, in the case of Exemplary embodiment 8, the VBUS may be allocated only to any one of pins A4 204a and B4 204b, the VBUS may be allocated to two pins (pins A4 and A9; pins A4 and B4) including pin A4 204a, or the VBUS may be allocated to two pins (pins A4 and B4; pins A9 and B4) including pin B4 (204b), and as illustrated in FIG. 19, the VBUS may be allocated to all of pins A4 204a, A9 209a, and B4 204b. In this case, among the pins of the electrode device 10 side terminal 100, a pin corresponding to a pin to which the PULL-DOWN of the measurement device 20 side terminal 200 is allocated and a pin corresponding to a pin to which the VBUS is allocated are allocated by the Short. More specifically, when the VBUS is allocated to pin A4 204a of the terminal 200, pins A4 104a and A9 109a of the terminal 100 are connected to each other by the short 130 as illustrated in FIG. 20A and when the VBUS is allocated to pin B4 204b of the terminal 200, pins B4 104b and B9 109b of the terminal 100 are connected to each other by the short 130 as illustrated in FIG. 20B. When the VBUS is allocated to pin A9 209a of the terminal 200 in Exemplary embodiment 8, pins A9 109a and B9 109b of the terminal 100 should not be connected to each other by the short. In this case, when pins A9 109a and B9 109b of the terminal 100 are connected to each other by the short, if the terminal 100 and the terminal 200 are connected in the forward direction or connected in the reverse direction, the signal value of the PULL-DOWN is not distinguished and the PULL-DOWN similarly has the high (1) signal value.

As described above, after allocation of D+/D−, VBUS, GND, and the determination signal (PULL-UP or PULL-DOWN) to the pins of the electrocardiogram measurement device 20 side terminal 200 is completed, electrode signals (V1 to V6, RA, LA, RL, and LL) detected through the plurality of electrodes 11a to 11j of the electrocardiogram electrode device 10 are allocated to the remaining pins. To this end, in the present invention, among at least 6 pairs of pins that are not allocated any of the D+/D−, VBUS, GND, and determination signal (PULL-UP or PULL-DOWN) of the terminal 200 of the electrocardiogram measurement device 20, electrode signals of V1 to V6 are allocated to pins belonging to three pairs, any two electrode signals of RA, LA, and LL are allocated to pins belonging to one pair, one remaining electrode signal which is not allocated among RA, LA, and LL is allocated to the pins belonging to one pair, and the electrode signal of RL is allocated to the pins belonging to one pair. Hereinafter, in the case of Exemplary embodiments 1 to 8 in which the determination signal (PULL-UP or PULL-DOWN) is exemplarily allocated to the pins according to the standard pin-out configuration of the USB Type-C terminal, a case where input electrode signals V1 to V6, RA, LA, RL, and LL are allocated to the remaining pins will be described.

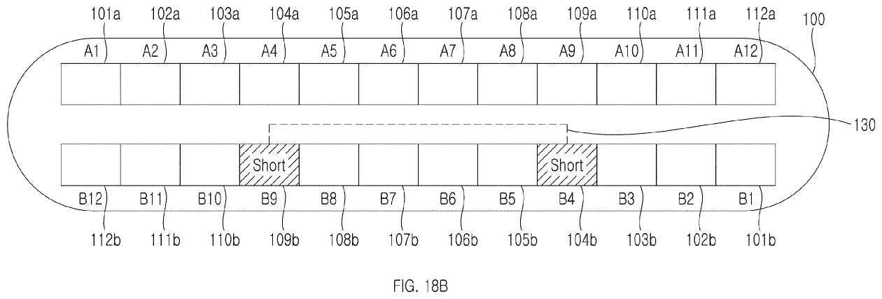

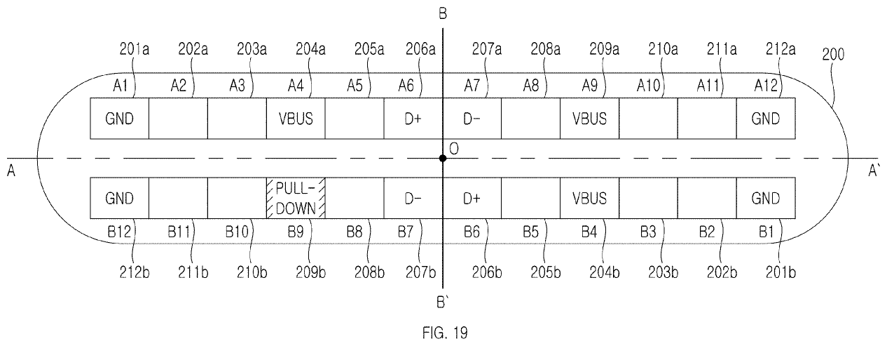

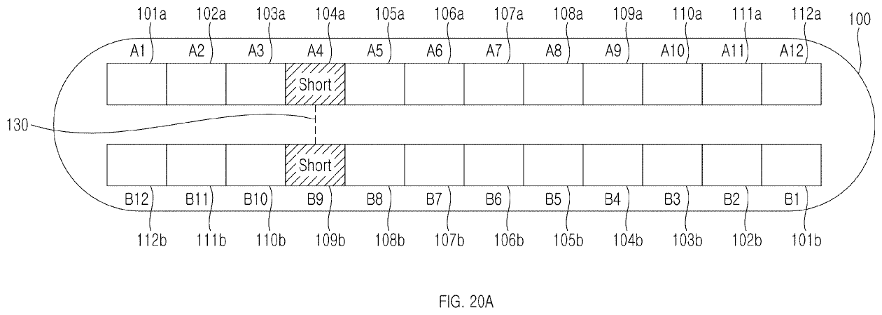

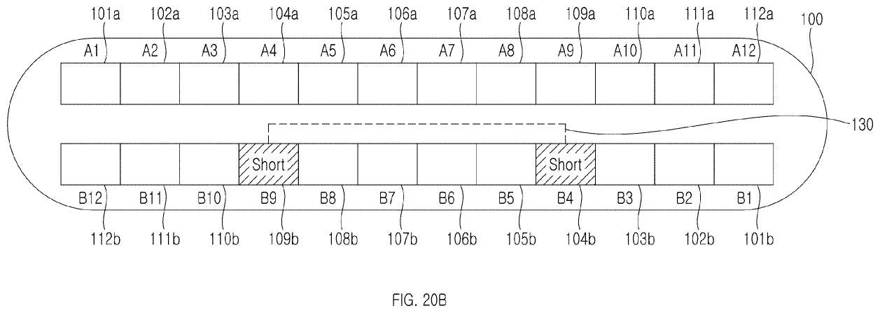

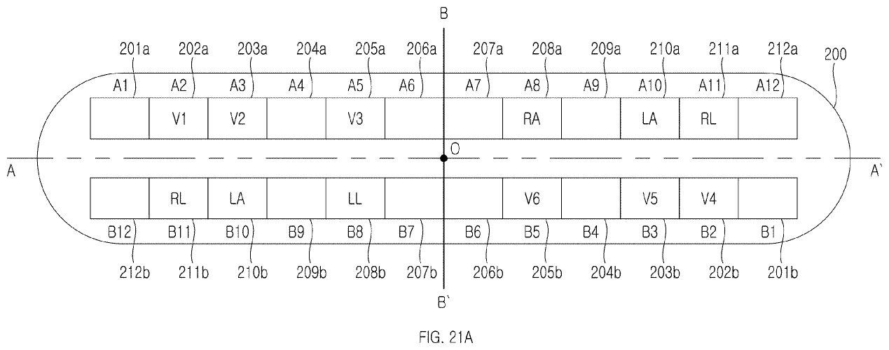

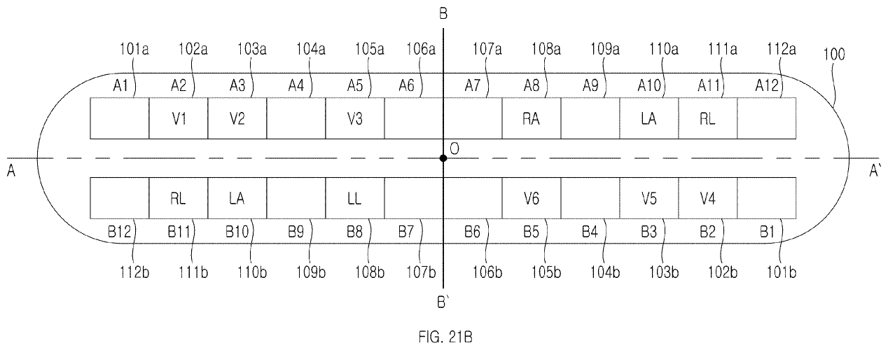

Among a total of 24 pins 201a to 212a and 201b of the channel electrocardiogram measurement device 20 side terminal 200, pins to which D+/D−, VBUS, GND, and PULL-UP are not allocated are a total of 12 pins of pin A2 202a, pin A3 203a, pin A5 205a, pin A8 208a, pin A10 210a, pin A11 211a, pin B2 202b, pin B3 203b, pin B5 205b, pin B8 208b, pin B10 210b, and pin B11 211b in Exemplary embodiments 1 to 8. In the present invention, 10 electrode signals (V1 to V6, RA, LA, RL, and LL) are allocated to the 12 pins. More specifically, except for pins to which D+/D−, VBUS, GND, and PULL-UP may be allocated among a total of 24 pins 201a to 212a and 201b of the channel electrocardiogram measurement device 20 side terminal 200, 6 pairs of pins ((pin A2, pin B2), (pin A3, pin B3), (pin A5, pin B5), (pin A8, pin B8), (pin A10, pin B10), and (pin A11, pin B11)) as the remaining pins to which the signal is not yet allocated remain and among 6 pairs of pins, 6 electrode signals of V1 to V6 are allocated to 6 pins, which are pins belonging to any 3 pairs, any two electrode signals ((RA, LA) or (RA, LL) or (LA, LL)) among RA, LA, and LL are allocated to each of two pins which are pins belonging to any one pair, and the remaining electrode signal which is not allocated above among RA, LA, and LL is allocated to at least any one of two pins which are pins belonging to any one pair. The electrode signal of RL is allocated to at least any one of two pins which are pins belonging to one pair which remain last. An exemplary embodiment of allocating the electrode signals (V1 to V6, RA, LA, RL, and LL) to the pins of the channel electrocardiogram measurement device 20 side terminal 200 is illustrated in FIG. 21A and is not limited thereto.

In response to allocating the electrode signals (V1 to V6, RA, LA, RL, and LL) to the pins of the electrocardiogram measurement device 20 side terminal 200, the electrode signals (V1 to V6, RA, LA, RL, and LL) are allocated to the pins of the electrocardiogram electrode device 10 side terminal 100. That is, among the pins of the electrocardiogram electrode device 10 side terminal 100, a pin corresponding to the pin to which the electrode signals (V1 to V6, RA, LA, RL, and LL) to the pins of the electrocardiogram measurement device 20 side terminal 200 are allocated is determined as an output pin of the electrode signals (V1 to V6, RA, LA, RL, and LL). As an example, in response to allocating the electrode signals (V1 to V6, RA, LA, RL, and LL) to the pins of the electrocardiogram measurement device 20 side terminal 200 as illustrated in FIG. 21A, the electrode signals (V1 to V6, RA, LA, RL, and LL) are allocated to the pins of the electrocardiogram electrode device 10 side terminal 100 as illustrated in FIG. 21B.

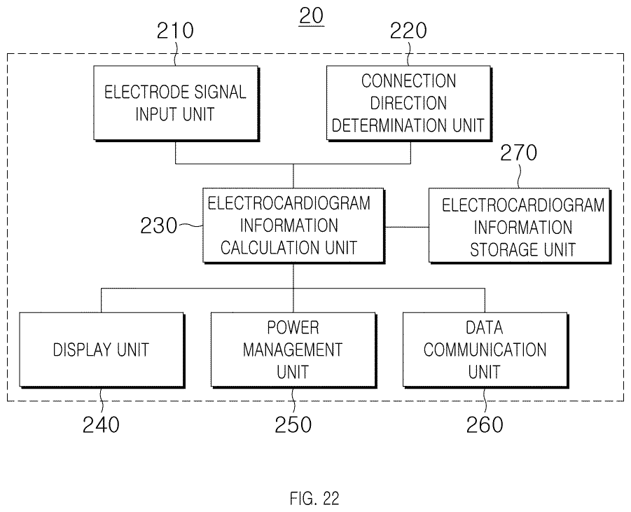

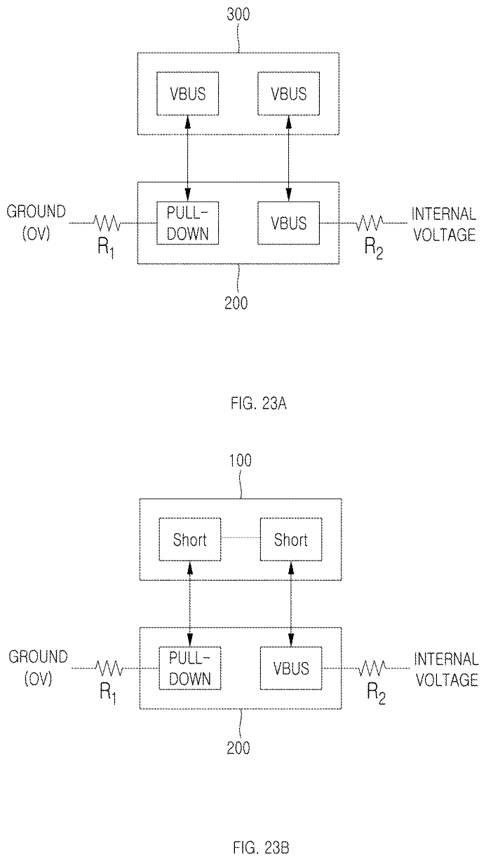

FIG. 22 is a block diagram for describing a configuration and an operation of a wearable electrocardiogram measurement device 20 with a nondirectional USB terminal according to the present invention.

Referring to FIG. 22, an electrocardiogram measurement device 20 having a nondirectional USB terminal 200 according to the present invention includes an electrode signal input unit 210, a connection direction determination unit 220, an electrocardiogram information calculation unit 230, a display unit 240, a power management unit 250, a data communication unit 260, and an electrocardiogram information storage unit 270.

The electrode signal input unit 210 receives electrode signals (V1 to V6, RA, RL, LA, and LL) sensed by the electrocardiogram electrode device 10 through the terminal 200. Here, signal values input into respective pins of the terminal 200 vary depending on a connection direction (forward direction or reverse direction) between the electrocardiogram measurement device 20 side terminal 200 and the electrocardiogram electrode device 10 side terminal 100.

The connection direction determination unit 220 determines whether the connection between the electrocardiogram measurement device 20 side terminal 200 and the electrocardiogram electrode device 10 side terminal 100 is in the forward direction or the reverse direction based on the determination signal (PULL-UP or PULL-DOWN). In the case that any one pin of the electrocardiogram measurement device 20 side terminal 200 is configured as a PULL-UP pin, when the electrode device 10 side terminal 100 is electrically connected to the measurement device 20 side terminal 200, if a state value of the PULL-UP is in a low (0) state, the connection direction determination unit 220 determines that the connection between the measurement device 20 side terminal 200 and the electrode device 10 side terminal 100 is in the forward direction. On the other hand, if the state value of the PULL-UP is in a high (1) state, the connection direction determination unit 220 determines that the connection between the measurement device 20 side terminal 200 and the electrode device 10 side terminal 100 is in the reverse direction. Meanwhile, in the case that any one pin of the electrocardiogram measurement device 20 side terminal 200 is configured as a PULL-DOWN pin, when the electrode device 10 side terminal 100 is electrically connected to the measurement device 20 side terminal 200, if the state value of the PULL-DOWN is in the high (1) state, the connection direction determination unit 220 determines that the connection between the measurement device 20 side terminal 200 and the electrode device 10 side terminal 100 is in the forward direction. On the other hand, if the state value of the PULL-DOWN is in the low (0) state, the connection direction determination unit 220 determines that the connection between the measurement device 20 side terminal 200 and the electrode device 10 side terminal 100 is in the reverse direction. More specifically, in the case of allocating the PULL-UP to the pin according to the standard pin-out configuration of the USB Type-C terminal (Exemplary embodiments 1 to 4), when the electrode device 10 side terminal 100 is connected to the measurement device 20 side terminal 200 in the forward direction, the pin of the measurement device 20 side terminal 200 allocated to the GND and any one pin of the pins of the electrode device 10 side terminal 100 allocated to the Short are electrically connected to each other. In this case, the other pin among the pins of the electrode device 10 side terminal 100 allocated the Short and the pin of the measurement device 20 side terminal 200 allocated to the PULL-UP are electrically connected to each other, and as a result, the state value of the PULL-UP is changed to the low (0) state. On the other hand, when the electrode device 10 side terminal 100 is connected to the measurement device 20 side terminal 20 in the reverse direction, the state value of the PULL-UP does not change and is maintained in the high (1) state. Meanwhile, in the case of allocating the PULL-DOWN to the pin according to the standard pin-out configuration of the USB Type-C terminal (Exemplary embodiments 5 to 8), when the electrode device 10 side terminal 100 is connected to the measurement device 20 side terminal 200 in the forward direction, the pin of the measurement device 20 side terminal 200 allocated to the VBUS and any one pin of the pins of the electrode device 10 side terminal 100 allocated to the Short are electrically connected to each other. In this case, the other pin among the pins of the electrode device 10 side terminal 100 allocated the Short and the pin of the measurement device 20 side terminal 200 allocated to the PULL-DOWN are electrically connected to each other, and as a result, the state value of the PULL-DOWN is changed to the high (1) state. On the other hand, when the electrode device 10 side terminal 100 is connected to the measurement device 20 side terminal 20 in the reverse direction, the state value of the PULL-DOWN does not change and is maintained in the low (0) state. As such, by checking the state value of the determination signal (PULL-UP or PULL-DOWN) that varies depending on the connection direction (forward or reverse direction) between the terminals, the connection direction determination unit 220 may determine whether the connection between the measurement device 20 side terminal 200 and the electrode device 10 side terminal 100 is in the forward direction or the reverse direction.

The electrocardiogram information calculation unit 240 combines the electrode signals V1 to V6, RA, RL, LA, and LL input through the terminal 200 based on the determination result of the connection direction determination unit 220 to acquire electrocardiogram information on the user wearing the electrocardiogram electrode device equipment 10. In addition, the electrocardiogram information calculation unit 240 may acquire cardiovascular state information (that is, average heart rate, maximum heart rate, minimum heart rate, instantaneous heart rate, etc.) of the user wearing the electrocardiogram electrode device 10 from the electrode signals V1 to V6, RA, RL, LA, and LL input through the terminal 200.

As an example, it is described that when the electrode signals V1 to V6, RA, RL, LA, and LL are allocated as illustrated in FIGS. 21A and 21B while the electrocardiogram measurement device 20 side terminal 200 and the electrocardiogram electrode device 10 side terminal 100 follow the standard pin-out configuration of the USB Type-C terminal, the electrocardiogram information calculation unit 240 measures the electrocardiogram information. When the electrode device 10 side terminal 100 is connected to the measurement device 20 side terminal 200 in the forward direction, the connection direction determination unit 220 determines that the connection between the terminals is in the forward direction, and as a result, the electrocardiogram information calculation unit 240 acquires the cardiogram information from input values of the electrode signals V1 to V6, RA, RL, LA, and LL allocated to the pins of the terminal 200 through calculation shown in Table 2 below.

TABLE 2

| | | |
|---|---|---|
| 12-channel | | |
| electrocardiogram | Calculation | Acquisition |
| information | value | value |

| | | | |
|---|---|---|---|
| Bipolar | Lead I | LA - RA | Lead I |
| standard guided | Lead II | RA - LL | Lead II |
| electrocardiogram | Lead III | LL - LA | Lead III |
| Unipolar | aVR | RA - WCT | aVR |
| limb guided | | (average value) | |
| electrocardiogram | aVL | LA - WCT | aVL |
| | | (average value) | |
| | aVF | LL - WCT | aVF |
| | | (average value) | |
| Unipolar | V1 | V1 - WCT | V1 |
| chest guided | | (average value) | |
| electrocardiogram | V2 | V2 - WCT | V2 |
| | | (average value) | |
| | V3 | V3 - WCT | V3 |
| | | (average value) | |
| | V4 | V4 - WCT | V4 |
| | | (average value) | |
| | V5 | V5 - WCT | V5 |
| | | (average value) | |
| | V6 | V6 - WCT | V6 |
| | | (average value) | |

As shown in Table 2, when the connection direction determination unit 220 determines that the connection between the terminals is in the forward direction, the electrocardiogram information calculation unit 240 acquires 12-channel electrocardiogram information derived from the calculation value as accurate electrocardiogram information as it is.

On the other hand, when the electrode device 10 side terminal 100 is connected to the measurement device 20 side terminal 200 in the reverse direction, the connection direction determination unit 220 determines that the connection between the terminals is in the reverse direction, and as a result, the electrocardiogram information calculation unit 240 calculates the electrocardiogram information from input values of the electrode signals V1 to V6, RA, RL, LA, and LL allocated to the pins of the terminal 200 as shown in Table 3 below and corrects the calculated electrocardiogram information to acquire the electrocardiogram information.

TABLE 3

| 12-channel | | | Acquisition value |
|---|---|---|---|
| electrocardiogram | | Calculation | (correction |
| information | | value | value) |
| Bipolar | Lead I | LA - RA | (Lead I) |
| standard guided | Lead II | RA - LL | (Lead II) |
| electrocardiogram | Lead III | LL - LA | (Lead III) |
| Unipolar | aVR | RA - WCT | aVL |
| limb guided | | (average value) | |
| electrocardiogram | aVL | LA - WCT | aVR |
| | | (average value) | |
| | aVF | LL - WCT | aVF |
| | | (average value) | |
| Unipolar | V1 | V1 - WCT | V4 |
| chest guided | | (average value) | |
| electrocardiogram | V2 | V2 - WCT | V5 |
| | | (average value) | |
| | V3 | V3 - WCT | V6 |
| | | (average value) | |
| | V4 | V4 - WCT | V1 |
| | | (average value) | |
| | V5 | V5 - WCT | V2 |
| | | (average value) | |
| | V6 | V6 - WCT | V3 |
| | | (average value) | |

As shown in Table 3, when the connection direction determination unit 220 determines that the connection between the terminals is in the reverse direction, the electrocardiogram information calculation unit 240 acquires accurate 12-channel electrocardiogram information by correcting the electrocardiogram information derived from the calculation value.

The display unit 240 is disposed on one surface of the electrocardiogram measurement device 20 to externally display the user's electrocardiogram information or cardiovascular state information acquired by the electrocardiogram information calculation unit 240. In addition, the display unit 240 may externally display power and battery status, electrode attachment status, and Bluetooth connection status by using not only the user's electrocardiogram information and cardiovascular state information, but also an LED array color and blinking speed. As a display device for implementing the display unit 240, for example, LCD, LED, etc., may be used, but the display device is not limited thereto.

The power management unit 250 receives external power through a power adapter (charger) connected to the terminal 200 to charge power of the battery or receives power from a battery to supply the power to respective components and manage a charge state of the battery. Further, the power management unit 250 may receive, from the battery, power for generating a state input value of the determination signal (PULL-UP or PULL-DOWN) as described below with reference to FIGS. 23A and 23B.

The data communication unit 260 transmits the user's electrocardiogram information or cardiovascular state information acquired by the electrocardiogram information calculation unit 240 to an external device (desktop, laptop, tablet, etc.) connected to the terminal 200 through wired communication. In some cases, the data communication unit 260 may transmit to an external server of a hospital/specialized institution through wireless communication. At this time, Ethernet communication may be used as a wired communication method, and as a wireless communication method, at least any one of WiFi, Zigbee, Bluetooth, RF, 3G, 4G, LTE, LTE-A, and Wireless Broadband Internet (WiBro) may be used to implement the data communication unit, but the present invention is not limited thereto.

The electrocardiogram information storage unit 270 stores the user's electrocardiogram information or cardiovascular state information acquired by the electrocardiogram information calculation unit 240. In this case, the electrocardiogram storage unit 270 may classify and store the electrocardiogram information or cardiovascular state information for each user who wears the electrocardiogram electrode device 10. The electrocardiogram information storage unit 270 may use, for example, a Secure Digital (SD) card, a micro SD card, or a flash memory as a memory for storing the electrocardiogram information or cardiovascular state information, but is not limited thereto.

On the other hand, as described above with reference to FIGS. 13 to 20, in order to configure the pin function of the terminal assembly using the PULL-DOWN in the present invention, as illustrated in FIGS. 23A and 23B, the battery mounted inside the electrocardiogram measurement device 20 and the pin allocated to the VBUS of the terminal 200 are electrically connected to each other via a resistor R2. The electrocardiogram measurement device 20 according to the present invention receives a VBUS output of a terminal 300 of the power adapter and supplies charging power to the battery through the connection between the pin to which the VBUS is allocated in the terminal 200 and the pin to which the VBUS is allocate din the terminal 300 of the power adapter (charger). In the present invention, in order to check the connection direction between the terminal 100 of the electrode device 10 and the terminal 200 of the measurement device 20 using the PULL-DOWN, a specific voltage should be applied to the pin to which the VBUS is allocated in the terminal 200. The reason is that when the terminal 100 of the electrode device 10 and the terminal 200 of the measurement device 20 are connected to each other in the forward direction, the state value of the PULL-DOWN should be changed to the high (1) state through the VBUS. As illustrated in FIG. 23B, by applying a voltage by a battery (for example, an internal voltage of 3.3V) mounted inside the electrode device 10 to the pin to which the VBUS is allocated in the terminal 200, a specific voltage may be applied to the pin to which the VBUS is allocated. When an internal voltage (e.g., 3.3 V) for applying a voltage to the VBUS to check the connection direction in the electrocardiogram measurement device 20 is different from a charging voltage (e.g., 5.0 V) applied to the VBUS through the power adapter (charger), if there is no resistor R2, different voltages are shorted in the VBUS at the time of connecting the terminal 200 of the measurement device 20 and the terminal 300 of the power adapter, and as a result, a short current may be generated. In order to prevent the generation of the short current, in the present invention, the specific voltage applied to the VBUS may be applied by using the resistor R2 in order to check the connection direction between the terminal 100 of the electrode device 10 and the terminal 200 of the measurement device 20 as illustrated in FIGS. 23A and 23B. In this case, a magnitude of the resistor R2 is sufficiently smaller than the magnitude of a resistor R1 on the PULL-DOWN side, so when the terminal 100 of the electrode device 10 is connected to the terminal 200 of the measurement device 20 in the forward direction, the state value of the PULL-UP has the high (1) state value by a sufficiently high voltage. Further, the magnitude of the resistor R2 is not too small, so when the terminal 300 of the power adapter is connected to the terminal 200 of the measurement device 20, the magnitude of current which flows inside is prevented from be too large for charging the battery.

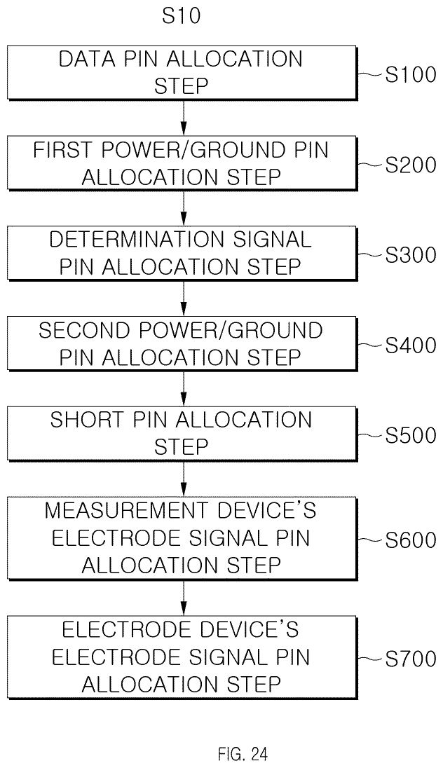

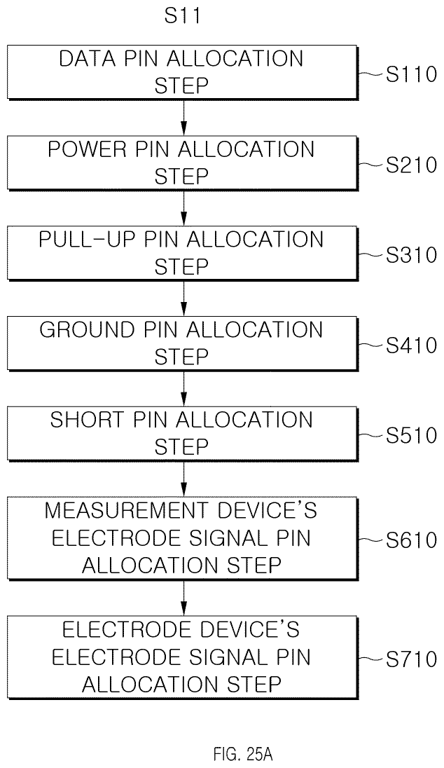

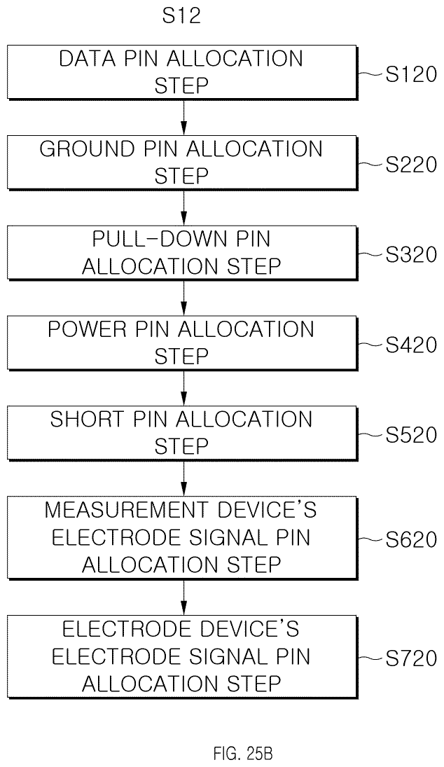

Hereinafter, a method for configuring a pin-out of a nondirectional USB terminal assembly for measuring an electrocardiogram according to the present invention will be described with reference to FIGS. 24 and 25.

Referring to FIG. 24, in the method for configuring a pin-out of a nondirectional USB terminal assembly for measuring an electrocardiogram according to the present invention (S10), D+/D− for data communication with an external device is first allocated to pins belonging to one pair among pins of the electrocardiogram measurement device 200 side terminal 20 (data pin allocation step, S100).

Next, among pins of the measurement device 200 side terminal 20 to which D+/D− is not allocated in step S100, any one of the VBUS for power supply and the GND is allocated to the pins belonging to one pair (first power/ground pin allocation step, S200).

In addition, among the pins of the measurement device 200 side terminal 20 to which any one of D+/D− and VBUS and GND is not allocated in steps S100 and S200, the determination signal (PULL-UP or PULL-DOWN) for determining the connection direction between the measurement device 20 side terminal 200 and the electrode device 10 side terminal 100 is allocated to any one pin (determination signal pin allocation step, S300).

Next, in step S300, the remaining one of the VBUS and the GND, which is not allocated is allocated to at least one pin of a pin located opposite the pin to which the determination signal (PULL-UP or PULL-DOWN) is allocated and a pin located symmetrically to the pin to which the determination signal (PULL-UP or PULL-DOWN) is allocated (second power/ground pin allocation step, S400).

In addition, among the pins of the electrocardiogram electrode device 10 side terminal 100, a pin corresponding to a pin to which the determination signal of the measurement device 20 side terminal 200 is allocated in step S300 and a pin corresponding to a pin allocated in step S400 are allocated by the Short (short pin allocation step, S500).

Next, among the pins of the measurement device 20 side terminal 200 to which the D+/D−, VBUS, GND, and determination signals are not allocated, electrode signals for measuring the electrocardiogram are allocated (measurement device's electrode signal pin allocation step, S600). At this time, if the number of electrocardiogram channels to be measured is 12, in step S600, among the pins of the measurement device 20 side terminal 200 to which D+/D−, VBUS, GND, and determination signals are not allocated, electrode signals of the first to sixth electrodes (V1 to V6) may be allocated to pins belonging to three pairs, any two electrode signals among RA, LA, and LL may be allocated to the pins belonging to one pair, and the electrode signal of RL may be allocated to the pin belonging to one pair.

Last, among the pins of the electrode device 10 side terminal 100, the electrode signal is allocated to a pin corresponding to the pin to which the electrode signal of the measurement device 20 side terminal 200 is allocated in step S600 (electrode device's electrode signal pin allocation step, S700). In this case, if the number of electrocardiogram channels to be measured is 12, in step S700, among the pins of the electrode device 10 side terminal 100, the electrode signals V1 to V6, RA, LA, RL, and LL may be allocated to the pins corresponding to the pins to which the electrode signals V1 to V6, RA, LA, RL, and LL of the measurement device 20 side terminal 200 are allocated, respectively.

FIG. 25A is a flowchart for describing a method for configuring pin-out of a nondirectional USB terminal assembly for measuring an electrocardiogram by using a PULL-UP.

Referring to FIG. 25A, in the method for configuring a pin-out of a nondirectional USB terminal assembly for measuring an electrocardiogram by using a PULL-UP (S11), D+/D− for data communication with an external device is first allocated to pins belonging to one pair among pins of the electrocardiogram measurement device 200 side terminal 20 (data pin allocation step, S110).

Next, among pins of the measurement device 200 side terminal 20 to which D+/D− is not allocated in step S110, the VBUS for power supply is allocated to the pins belonging to one pair (power pin allocation step, S210).

In addition, among the pins of the measurement device 200 side terminal 20 to which any one of D+/D− and VBUS and GND is not allocated in steps S110 and S210, the PULL-UP for determining the connection direction between the measurement device 20 side terminal 200 and the electrode device 10 side terminal 100 is allocated to any one pin (pull-up pin allocation step, S310).

In addition, the GND is allocated to at least one of a pin located to face a pin to which the pull-up of the measurement device 20 side terminal 200 is allocated and a pin located symmetrically to the pin to which the pull-up is allocated in step S310 (ground pin allocation step, S410).

In addition, among the pins of the electrocardiogram electrode device 10 side terminal 100, a pin corresponding to a pin to which the PULL-UP of the measurement device 20 side terminal 200 is allocated in step S310 and a pin corresponding to a pin to which the GND is allocated in step S410 are allocated by the Short (short pin allocation step, S510).

Next, among the pins of the measurement device 20 side terminal 200 to which the D+/D−, VBUS, GND, and determination signals are not allocated, electrode signals for measuring the electrocardiogram are allocated (measurement device's electrode signal pin allocation step, S610). At this time, if the number of electrocardiogram channels to be measured is 12, in step S610, among the pins of the measurement device 20 side terminal 200 to which D+/D−, VBUS, GND, and determination signals are not allocated, electrode signals of the first to sixth electrodes (V1 to V6) may be allocated to pins belonging to three pairs, any two electrode signals among RA, LA, and LL may be allocated to the pins belonging to one pair, and the electrode signal of RL may be allocated to the pin belonging to one pair.

Last, among the pins of the electrode device 10 side terminal 100, the electrode signal is allocated to a pin corresponding to the pin to which the electrode signal of the measurement device 20 side terminal 200 is allocated in step S610 (electrode device's electrode signal pin allocation step, S710). In this case, if the number of electrocardiogram channels to be measured is 12, in step S710, among the pins of the electrode device 10 side terminal 100, the electrode signals V1 to V6, RA, LA, RL, and LL may be allocated to the pins corresponding to the pins to which the electrode signals V1 to V6, RA, LA, RL, and LL of the measurement device 20 side terminal 200 are allocated, respectively.

FIG. 25B is a flowchart for describing a method for configuring pin-out of a nondirectional USB terminal assembly for measuring an electrocardiogram by using a PULL-DOWN.

Referring to FIG. 25B, in the method for configuring a pin-out of a nondirectional USB terminal assembly for measuring an electrocardiogram by using a PULL-DOWN (S11), D+/D− for data communication with an external device is first allocated to pins belonging to one pair among pins of the electrocardiogram measurement device 200 side terminal 20 (data pin allocation step, S120).

Next, among pins of the measurement device 200 side terminal 20 to which D+/D− is not allocated in step S120, the GND is allocated to the pins belonging to one pair (ground pin allocation step, S220).

In addition, among the pins of the measurement device 200 side terminal 20 to which any one of D+/D− and GND is not allocated in steps S120 and S220, the PULL-DOWN for determining the connection direction between the measurement device 20 side terminal 200 and the electrode device 10 side terminal 100 is allocated to any one pin (pull-down pin allocation step, S320).

Next, the VBUS for power supply is allocated to at least one of a pin located to face a pin to which the pull-down is allocated and a pin located symmetrically to the pin to which the pull-down is allocated in step S320 (power pin allocation step, S420).

In addition, among the pins of the electrocardiogram electrode device 10 side terminal 100, a pin corresponding to a pin to which the PULL-DOWN of the measurement device 20 side terminal 200 is allocated in step S320 and a pin corresponding to a pin to which the VBUS is allocated in step S420 are allocated by the Short (short pin allocation step, S520).

Next, among the pins of the measurement device 20 side terminal 200 to which the D+/D−, VBUS, GND, and determination signals are not allocated, electrode signals for measuring the electrocardiogram are allocated (measurement device's electrode signal pin allocation step, S620). At this time, if the number of electrocardiogram channels to be measured is 12, in step S620, among the pins of the measurement device 20 side terminal 200 to which D+/D−, VBUS, GND, and determination signals are not allocated, electrode signals of the first to sixth electrodes (V1 to V6) may be allocated to pins belonging to three pairs, any two electrode signals among RA, LA, and LL may be allocated to the pins belonging to one pair, and the electrode signal of RL may be allocated to the pin belonging to one pair.

Last, among the pins of the electrode device 10 side terminal 100, the electrode signal is allocated to a pin corresponding to the pin to which the electrode signal of the measurement device 20 side terminal 200 is allocated in step S620 (electrode device's electrode signal pin allocation step, S720). In this case, if the number of electrocardiogram channels to be measured is 12, in step S720, among the pins of the electrode device 10 side terminal 100, the electrode signals V1 to V6, RA, LA, RL, and LL may be allocated to the pins corresponding to the pins to which the electrode signals V1 to V6, RA, LA, RL, and LL of the measurement device 20 side terminal 200 are allocated, respectively.

As described above, the optimal embodiment is disclosed in the drawings and the specification. Although specific terms have been used herein, the terms are only used for the purpose of describing the present invention and are not used to limit the scope of the present invention as defined in the claims. Therefore, it will be appreciated by those skilled in the art that various modifications and other embodiments equivalent thereto can be made therefrom. Accordingly, the true technical scope of the present invention should be defined by the technical spirit of the appended claims.

Further, the term “unit” used in the specification means software and hardware components such as field programmable gate array (FPGA) or ASIC and the “unit” performs predetermined roles. However, the “unit” is not a meaning limited to software or hardware. The “unit” may be configured to reside on an addressable storage medium and may be configured to play back one or more processors. Accordingly, as one example, the “unit” includes components such as software components, object oriented software components, class components, and task components, processes, functions, attributes, procedures, subroutines, segments of a program patent code, drivers, firmware, microcodes, circuitry, data, databases, data structures, tables, arrays, and variables.

Functions provided in the components and the “units” may be combined into a smaller number of components and “units” or separated into additional components and “units” and implemented to be driven by one or more CPUs in the device.

What is claimed is:

1. A method for configuring pin-out of a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, the method comprising:
a data pin allocation step of allocating D+/D− for data communication with the external device to pins belonging to one pair among the pins of the electrocardiogram measurement device-side terminal;
a power pin allocation step of allocating among pins of the measurement device-side terminal to which D+/D− is not allocated, the VBUS for power supply to the pins belonging to one pair;
a pull-up pin allocation step of allocating among pins to which the D+/D− and the VBUS of the measurement device-side terminal are not allocated, a PULL-UP to any one pin;
a ground pin allocation step of allocating GND to at least one of a pin located to face a pin to which the pull-up of the measurement device-side terminal is allocated and a pin located symmetrically to the pin to which the pull-up is allocated;
a short pin allocation step of allocating, among the pins of the electrode device-side terminal, a pin corresponding to a pin to which the PULL-UP of the measurement device-side terminal is allocated and a pin corresponding to a pin to which the GND by a Short;
a measurement device's electrode signal pin allocation step of allocating, among the pins of the measurement device-side terminal to which the D+/D−, VBUS, GND, and PULL-UP are not allocated, electrode signals for measuring the electrocardiogram; and
an electrode device's electrode signal pin allocation step of allocating, among the pins of the electrode device-side terminal, the electrode signal to the pin corresponding to the pin to which the electrode signal of the measurement device-side terminal is allocated.

2. The method of claim 1, wherein in the measurement device's electrode signal pin allocation step, among at least 6 pairs of pins that are not allocated any of the D+/D−, VBUS, GND, and determination signal (PULL-UP or PULL-DOWN) of the electrocardiogram measurement device-side terminal, electrode signals of V1 to V6 are allocated to pins belonging to three pairs, any two electrode signals of RA, LA, and LL are allocated to pins belonging to one pair, one remaining electrode signal which is not allocated among RA, LA, and LL is allocated to the pins belonging to one pair, and the electrode signal of RL is allocated to the pins belonging to one pair, and
in the electrode device's electrode signal pin allocation step, among the pins of the electrode device-side terminal, the electrode signals of V1 to V6, RA, RL, LA, and LL are allocated to pins corresponding to the pins to which the electrodes signals of V1 to V6, RA, RL, LA, and LL of the measurement device-side terminal are allocated.

3. The method of claim 2, wherein the measurement device-side terminal is a USB Type-C female terminal (socket) in which pins A1 to A12 and pins B1 to B12 are arranged in two rows,
the measurement device-side terminal is a USB Type-C male terminal (plug) in which pins A1 to A12 and pins B1 to B12 are arranged in two rows,
when the USB Type-C female terminal and the USB Type-C female terminal are connected in a forward direction, pins A1 to A12 and pins B1 to B12 of the USB Type-C female terminal and pins A1 to A12 and pins B1 to B12 of the USB Type-C female terminal are shorted to correspond to each other in order, and
when the USB Type-C female terminal and the USB Type-C female terminal are connected in a reverse direction, pins A1 to A12 and pins B1 to B12 of the USB Type-C female terminal and pins B1 to B12 and pins A1 to A12 of the USB Type-C female terminal are shorted to correspond to each other in order.

4. The method of claim 3, wherein in the data pin allocation step, D+ is allocated to pins A6 and B6 diagonally located in the USB Type-C female terminal and D+ is allocated to pins A7 and B7.

5. The method of claim 4, wherein in the power pin allocation step, the VBUS is allocated to pins A4, A9, B4, and B9 in the USB Type-C female terminal.

6. The method of claim 5, wherein in the pull-up pin allocation step, the PULL-UP is allocated to any one pin of pins A1, A12, B1, and B12 in the USB Type-C female terminal.

7. The method of claim 6, wherein in the pull-up pin allocation step, the PULL-UP is allocated to pin A1 in the USB Type-C female terminal,
in the ground pin allocation step, the GND is allocated to at least one of pin A12 or pin B12 in the USB Type-C female terminal, and
in the short pin allocation step, pins A1 and A12 are allocated by the short to each other or pins Aland B12 are allocated by the short to each other in the USB Type-C male terminal.

8. The method of claim 6, wherein in the pull-up pin allocation step, the PULL-UP is allocated to pin A12 in the USB Type-C female terminal,
in the ground pin allocation step, the GND is allocated to at least one of pin A1 or pin B1 in the USB Type-C female terminal, and
in the short pin allocation step, pins A1 and A12 are allocated by the short to each other or pins A12 and Blare allocated by the short to each other in the USB Type-C male terminal.

9. The method of claim 6, wherein in the pull-up pin allocation step, the PULL-UP is allocated to pin B1 in the USB Type-C female terminal,
in the ground pin allocation step, the GND is allocated to at least one of pin A12 or pin B12 in the USB Type-C female terminal, and
in the short pin allocation step, pins A12 and B1 are allocated by the short to each other or pins B1 and B12 are allocated by the short to each other in the USB Type-C male terminal.

10. The method of claim 6, wherein in the pull-up pin allocation step, the PULL-UP is allocated to pin B12 in the USB Type-C female terminal,
in the ground pin allocation step, the GND is allocated to at least one of pin A1 or pin B1 in the USB Type-C female terminal, and
in the short pin allocation step, pins A1 and B12 are allocated by the short to each other or pins B1 and B12 are allocated by the short to each other in the USB Type-C male terminal.

11. An electrocardiogram measurement device having a terminal in which a pin is configured by a method of claim 1, the device comprising:
an electrode signal input unit receiving electrode signal sensed by the electrocardiogram electrode device through a terminal on the side of the measurement device;
a connection direction determination unit determining that a connection between the measurement device-side terminal and the electrode device-side terminal is in a forward direction when the PULL-UP is in a low state and determining that the connection between the measurement device-side terminal and the electrode device-side terminal is in a reverse direction when the PULL-UP is in a high state;
an electrocardiogram information calculation unit acquiring electrocardiogram information by combining the electrode signals based on a determination result of the connection direction determination unit; and
a display unit displaying the electrocardiogram information acquired by the electrocardiogram information calculation unit.

12. A method for configuring pin-out of a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, the method comprising:
a data pin allocation step of allocating D+/D− for data communication with the external device to pins belonging to one pair among the pins of the electrocardiogram measurement device-side terminal;
a ground pin allocation step of allocating, among pins of the measurement device-side terminal to which D+/D− is not allocated, the GND to the pins belonging to one pair;
a pull-up pin allocation step of allocating, among pins of the measurement device-side terminal to which the D+/D− and the GND are not allocated, a PULL-DOWN to any one pin;
a power pin allocation step of allocating VBUS for power supply to at least one of a pin located to face a pin to which the pull-down of the measurement device-side terminal is allocated and a pin located symmetrically to the pin to which the pull-down is allocated;
a short pin allocation step of among the pins of the electrode device-side terminal, a pin corresponding to a pin to which the PULL-DOWN of the measurement device-side terminal is allocated and a pin corresponding to a pin to which the VBUS is allocated by a Short;
a measurement device's electrode signal pin allocation step of allocating, among the pins of the measurement device-side terminal to which the D+/D−, VBUS, GND, and PULL-DOWN are not allocated, electrode signals for measuring the electrocardiogram; and
an electrode device's electrode signal pin allocation step of allocating, among the pins of the electrode device-side terminal, the electrode signal to the pin corresponding to the pin to which the electrode signal of the measurement device-side terminal is allocated.

13. The method of claim 12, wherein in the measurement device's electrode signal pin allocation step, among at least 6 pairs of pins that are not allocated any of the D+/D−, VBUS, GND, and PULL-DOWN of the electrocardiogram measurement device-side terminal, electrode signals of V1 to V6 are allocated to pins belonging to three pairs, any two electrode signals of RA, LA, and LL are allocated to pins belonging to one pair, one remaining electrode signal which is not allocated among RA, LA, and LL is allocated to the pins belonging to one pair, and the electrode signal of RL is allocated to the pins belonging to one pair, and
in the electrode device's electrode signal pin allocation step, among the pins of the electrode device-side terminal, the electrode signals of V1 to V6, RA, RL, LA, and LL are allocated to pins corresponding to the pins to which the electrodes signals of V1 to V6, RA, RL, LA, and LL of the measurement device-side terminal are allocated.

14. The method of claim 13, wherein the measurement device-side terminal is a USB Type-C female terminal (socket) in which pins A1 to A12 and pins B1 to B12 are arranged in two rows,
the measurement device-side terminal is a USB Type-C male terminal (plug) in which pins A1 to A12 and pins B1 to B12 are arranged in two rows,
when the USB Type-C female terminal and the USB Type-C female terminal are connected in a forward direction, pins A1 to A12 and pins B1 to B12 the USB Type-C female terminal and pins A1 to A12 and pins B1 to B12 of the USB Type-C female terminal are shorted to correspond to each other in order, and
when the USB Type-C female terminal and the USB Type-C female terminal are connected in a reverse direction, pins A1 to A12 and pins B1 to B12 the USB Type-C female terminal and pins B1 to B12 and pins A1 to A12 of the USB Type-C female terminal are shorted to correspond to each other in order.

15. The method of claim 14, wherein in the data pin allocation step, D+ is allocated to pins A6 and B6 diagonally located in the USB Type-C female terminal and D+ is allocated to pins A7 and B7.

16. The method of claim 15, wherein in the ground power pin allocation step, the GND is allocated to pins A1, A12, B1, and B12 in the USB Type-C female terminal.

17. The method of claim 16, wherein in the pull-down pin allocation step, the PULL-DOWN is allocated to any one pin of pins A4, A9, B4, and B9 in the USB Type-C female terminal.

18. The method of claim 17, wherein in the pull-down pin allocation step, the PULL-DOWN is allocated to pin A4 in the USB Type-C female terminal,
in the power pin allocation step, the VBUS is allocated to at least one of pin A9 or pin B9 in the USB Type-C female terminal, and
in the short pin allocation step, pins A4 and A9 are allocated by the short to each other or pins A4 and B9 are allocated by the short to each other in the USB Type-C male terminal.

19. The method of claim 17, wherein in the pull-down pin allocation step, the PULL-DOWN is allocated to pin A9 in the USB Type-C female terminal,
in the power pin allocation step, the VBUS is allocated to at least one of pin A4 or pin B4 in the USB Type-C female terminal, and
in the short pin allocation step, pins A4 and A9 are allocated by the short to each other or pins A9 and B4 are allocated by the short to each other in the USB Type-C male terminal.

20. The method of claim 17, wherein in the pull-down pin allocation step, the PULL-DOWN is allocated to pin B4 in the USB Type-C female terminal,
in the power pin allocation step, the VBUS is allocated to at least one of pin A9 or pin B9 in the USB Type-C female terminal, and
in the short pin allocation step, pins A9 and B4 are allocated by the short to each other or pins B4 and B9 are allocated by the short to each other in the USB Type-C male terminal.

21. The method of claim 17, wherein in the pull-down pin allocation step, the PULL-DOWN is allocated to pin B9 in the USB Type-C female terminal,
in the power pin allocation step, the VBUS is allocated to at least one of pin A4 or pin B4 in the USB Type-C female terminal, and
in the short pin allocation step, pins A4 and B9 are allocated by the short to each other or pins B4 and B9 are allocated by the short to each other in the USB Type-C male terminal.

22. An electrocardiogram measurement device having a terminal in which a pin is configured by a method of claim 12, the device comprising:
an electrode signal input unit receiving electrode signal sensed by the electrocardiogram electrode device through a terminal on the side of the measurement device;
a connection direction determination unit determining that a connection between the measurement device-side terminal and the electrode device-side terminal is in a forward direction when the PULL-DOWN is in a high state and determining that the connection between the measurement device-side terminal and the electrode device-side terminal is in a reverse direction when the PULL-DOWN is in a low state;
an electrocardiogram information calculation unit acquiring electrocardiogram information by combining the electrode signals based on a determination result of the connection direction determination unit; and
a display unit displaying the electrocardiogram information acquired by the electrocardiogram information calculation unit.

23. A method for configuring pin-out of a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, the method comprising:
a data pin allocation step of allocating D+/D− for data communication with the external device to pins belonging to one pair among the pins of the electrocardiogram measurement device-side terminal;
a first power/ground pin allocation step of allocating, among pins to which D+/D− of the measurement device-side terminal is not allocated, anyone of VBUS for power supply and GND to the pins belonging to one pair;
a determination signal pin allocation step of allocating a determination signal for determining the connection direction between the measurement device-side terminal and the electrode device-side terminal to at least one pin among pins to which the D+/D− and any one of the VBUS and the GND of the measurement device-side terminal are not allocated;
a second power/ground pin allocation step of allocating the remaining one which is not allocated of the VBUS and the GND to at least one of a pin located to face the pin to which the determination signal of the measurement device-side terminal is allocated and a pin located symmetrically to the pin to which the determination signal is allocated;
a short pin allocation step of allocating, among the pins of the electrode device-side terminal, a pin corresponding to the pin to which the determination signal of the measurement device-side terminal is allocated and a pin corresponding to the pin allocated in the second power/ground pin allocation step by a Short;
a measurement device's electrode signal pin allocation step of allocating, among the pins of the measurement device-side terminal to which the D+/D−, VBUS, GND, and PULL-UP are not allocated, electrode signals for measuring the electrocardiogram; and
an electrode device's electrode signal pin allocation step of allocating, among the pins of the electrode device-side terminal, the electrode signal to the pin corresponding to the pin to which the electrode signal of the measurement device-side terminal is allocated.

24. An electrocardiogram measurement device having a terminal in which a pin is configured by the method of claim 23, the device comprising:
an electrode signal input unit receiving electrode signal sensed by the electrocardiogram electrode device through a terminal on the side of the measurement device;
a connection direction determination unit determining whether a connection between the measurement device-side terminal and the electrode device-side terminal is in a forward direction or a reverse direction based on the determination signal;
an electrocardiogram information calculation unit acquiring electrocardiogram information by combining the electrode signals based on a determination result of the connection direction determination unit; and
a display unit displaying the electrocardiogram information acquired by the electrocardiogram information calculation unit.

25. An electrocardiogram electrode device having a nondirectional USB terminal assembly capable of measuring an electrocardiogram regardless of a connection direction between an electrocardiogram electrode device-side terminal in which two pins located diagonally to each other are arranged in two rows with one pair and an electrocardiogram measurement device-side terminal in which pins corresponding to the pins of the measurement device-side terminal, respectively are arranged in two rows, the device comprising:
an electrode module constituted by a plurality of electrodes attachable to a body of a user; and
a nondirectional USB terminal electrically connected to an electrocardiogram measurement device for transmitting electrode signal sensed through each of the plurality of electrodes,
wherein in the electrode device-side terminal, pins corresponding to pins of the measurement device-side terminal, respectively are arranged in two rows,
at least two pins among the pins of the electrode device-side terminal are shorted to each other, and
the electrode signals are allocated to pins which are not shorted to each other among the pins of the electrode device-side terminal.
